(19) United States
(12) Patent Application Publication
(10) Pub. No.: US 2024/0252552 A1
(43) Pub. Date: Aug. 1, 2024

(54) LIPOTEICHOIC ACID FROM STAPHYLOCOCCUS EPIDERMIDIS AND ITS USE TO MODULATE NEURONAL ACTIVITY VIA PD-1 INHIBITORY SIGNALLING IN NEURONS

(71) Applicant: Northwestern University, Evanston, IL (US)

(72) Inventor: Praveen Thumbikat, Evanston, IL (US)

(21) Appl. No.: 18/418,897

(22) Filed: Jan. 22, 2024

Related U.S. Application Data
(60) Provisional application No. 63/480,881, filed on Jan. 20, 2023.

(51) Int. Cl. A61K 35/74; A61K 31/7032; A61P 25/04

(52) U.S. Cl. CPC A61K 35/74; A61K 31/7032; A61P 25/04

(57) ABSTRACT

Disclosed herein are compositions comprising lipoteichoic acid (LTA) and the use thereof for modulating neuronal activity. The disclosed LTA compositions may be prepared using LTA that is derived from the Gram positive bacteria Staphylococcus epidermidis. The disclosed LTA compositions may be administered to treat pain, including but not limited to chronic pelvic pain syndrome (CPPS), neurogenic pain, or neurogenic inflammation via PD-1 inhibitory signaling in neurons. The disclosed LTA compositions may be administered in order to modulate the expression of endogenous opioids.

### CROSS-REFERENCE TO RELATED APPLICATIONS

The present application claims priority to U.S. Provisional Patent Application No. 63/480,881 that was filed Jan. 20, 2023. The entire contents of which are hereby incorporated by reference.

### STATEMENT REGARDING FEDERALLY SPONSORED RESEARCH OR DEVELOPMENT

This invention was made with government support under DK108127 awarded by the National Institutes of Health. The government has certain rights in the invention.

### FIELD OF THE INVENTION

The field of the invention relates to compositions comprising lipoteichoic acid (LTA) and the use thereof for modulating neuronal activity. The disclosed LTA compositions may be prepared using LTA that is derived from the Gram-positive bacteria Staphylococcus epidermidis. The disclosed LTA compositions may be administered to treat pain, including but not limited to chronic pelvic pain syndrome (CPPS) via PD-1 inhibitory signaling in neurons. The disclosed LTA compositions may be administered in order to modulate the expression of endogenous opioids.

### BACKGROUND

Staphylococcus epidermidis (SE) is a commensal bacterium of the skin that has a role in mediating local immune homeostasis. (See U.S. Publication No. 2018/0280419, the content of which is incorporated herein by reference in its entirety). During local immune activation SE can dampen inflammation signals. The cell wall lipoteichoic acid of SE (SELTA) is known to be a ligand for TLR2 which mediates signaling responses by forming homo or hetero dimers with TLR1 and/or TLR6.

### SUMMARY

Here, the inventor demonstrates that Staphylococcus epidermidis lipoteichoic acid (SELTA) can modulate the nociceptive threshold of primary sensory neurons through activation of the recently identified PD-1 pathway of neuronal homeostasis. The data suggests that SELTA acts as an exogenous ligand of this novel pathway to mediate its anti-nociceptive effects. In addition to this, LTA activates expression of endogenous opioid molecules as well as their cognate opioid receptor on neurons. These data show that LTA may serve as a disease modifying, antinociceptive, non-opioid molecule with therapeutic applications in chronic pain.

In addition to chronic nociceptive pain, the inventor believe that the disclosed methods are effective in the treatment of a variety of pain conditions, including neurogenic pain. For example, chemotherapy induced pain, multiple sclerosis (MS) induced pain, pain secondary to diabetic neuropathy, phantom limb and any associated pain.

### BRIEF DESCRIPTION OF THE FIGURES

The patent or application file contains at least one drawing executed in color. Copies of this patent or patent application publication with color drawing(s) will be provided by the Office upon request and payment of the necessary fee.

FIGS. 1A, 1B, and 1C. Intraurethral treatment of EAP mice with SELTA ameliorates pelvic tactile allodynia. (a) Pelvic tactile allodynia response frequencies, in Naïve, EAP and EAP with LTA treatment at day 21, in C57BL/6 mice, every 7 days for 28 days. Responses are depicted as increased percentage response frequency above baseline. (b) Pelvic tactile allodynia response change following treatment with LTA, using day 21 as baseline. (c) Response change frequencies from baseline (day 0) after 21 days of EAP development. Values for C57BL/6 mice with EAP, EAP & PBS (vehicle control), EAP & SELTA (S. epidermidis), EAP & SELTA II (second batch), EAP & SALTA (S. aureus) and EAP & BSLTA (B. subtilis) are shown, before and 7 days after LTA.

FIG. 2. SELTA requires TLR2 and activates NFkB in a dose dependent manner. SELTA induces NFkB activation in the reporter cell lines HEK-TLR2 but not in HEK-NULL (left panel). NFkB activity in response to SELTA is increased in a dose dependent manner in the THP-1 human monocytic reporter cell line (right panel).

FIG. 3. Pilot structural characterization of SELTA identified unique characteristics. ¹H NMR, glycosyl and fatty acid composition were performed on pooled C8-sepharose purified fractions of SELTA. Experiments were performed at the University of Georgia Complex Carbohydrate Research center.

FIGS. 4A, 4B, 4C, and 4D. SELTA instillation elevates PD-1 immunoreactivity in the nerve fibers of the prostate (A and B). PD-1 (red) immunoreactivity colocalized with the nerve fibers marked with BIII-tubulin (green) (C57BL/6 mice at day 28 of EAP, 7-days post-SELTA treatment). High magnification of the boxed region (C&D) shows the details of the colocalization of PD-1 and βIII-tubulin immunoreactivity (white arrows).

FIGS. 5A, 5B, 5C, 5D, and 5E. SELTA induced PD-1 expression in the DRG (A) In DRG ex vivo experiment, PD-1(green)immunoreactivity was evaluated in control and 100 ng/ml SELTA incubation for 24 hrs. Scale bars=10 μm. (B) Distribution of intensity of PD-1 immunoreactivity in DRG based on the size of individual neurons in control (upper) and SELTA treatment (bottom). (C&D) Cell-size differential and NeuN-normalized quantification of PD-1 immunoreactivity in DRG in control and SELTA treatment (E) QPCR shows PD-1 mRNA expression in control, SELTA, and MMG11+ SELTA. For all quantification analysis, a two-way ANOVA was used. *P<0.05, **P<0.01, #P<0.05. n=3 to 4 per group.

FIGS. 6A, 6B, and C. SELTA-induced phosphorylation of PD-1 in sensory neurons. Images show the phosphorylation of PD-1 (green) in neurons labeled with NeuN (red) in control (upper panel) and 100 ng/ml SELTA treatment (bottom panel) for 3 hrs. Quantification of phos-PD-1(B) alone or normalized to NeuN expressing cells (C), n=20-60 cells from 6-8 confocal images per group.

FIG. 7. SELTA-induced phosphorylation of SHP-1 in TG neurons. Images show the phosphorylation of SHP-1 (green) in control or SELTA treated cultured TG neurons.

FIG. 8. SELTA inhibits ATP-induced Ca2+ influx in cultured TG neurons and activity is blocked by inhibition of TLR2 or PD-1 signaling. Representative change in ratio of F340/F380 traces from single experiments and averaged data from three independent experiments is shown from fura-2 AM loaded TG neurons exposed to 100 ng/ml SELTA (Left panel) SELTA inhibits ATP-induced Ca2+ influx. (Middle panel)) Blocking TLR2 by MMG11 reverses SELTA-induced reduction of Ca2+ influx. (D) Blocking PD-1 using 100 ug/ml but not 10 ug/ml anti-PD-1 neutralizing antibody indicated that the inhibitory effect by SELTA was abrogated. Analysis with Student's t test, p<0.0001.

FIG. 9. SELTA-induced expression of PDYN in dorsal root ganglion. In an ex vivo cultured preparation, PDYN immunoreactivity increased with SELTA induction in DRG (bottom panel) as compared with control group (upper panel). Note that the increased PDYN expression was predominantly seen in the small- and medium-size afferent neurons as indicated by yellow arrows. Scale bar=20 μm.

FIG. 10. SELTA-induced elevated expression of KOR in dorsal root ganglion. In an ex vivo cultured preparation, KOR immunoreactivity increased with SELTA induction in DRG (bottom panels) as compared with control group (upper panels). Note that the increased KOR expression predominantly seen in the small- and medium-size afferent neurons as indicated by yellow arrows (bottom panel). Scale bar=20 μm.

FIG. 11 shows a schematic of SELTA based on empirical evidence.

FIG. 12 shows a schematic workflow for the purification of SELTA from S. epidermidis.

FIG. 13 shows a hypothetical model demonstrating a potential model for immunoregulation by SELTA.

FIG. 14 shows reduced FOXP3 mRNA in urine from men suffering from chronic prostatitis as compared to control which may be indicative of an immune imbalance in these subjects.

FIG. 15 shows increased methylation of IL10 gene in CPPS patients (black bar) compared to control which is correlated with reduced IL10 expression.

FIG. 16 shows a heatmap representation of the results presented in FIG. 15.

FIG. 17 shows dose dependent effects of SE-LTA on innate immune activation (NFkB) in THP-1 reporter cells.

FIG. 18 shows dose dependent effects of SELTA on innate immune activation (NFkB) in HEK-TLR2 vs HEK-NULL reporter cells. SELTA activity is not abolished by proteinase K treatment

FIG. 19 shows that autoimmune prostatitis (EAP) model demonstrated efficacy of LTA to dampen pain responses similar to NPI strain of S. epidermidis.

FIG. 20 shows a reduction in allodynia during EAP in mice administered SELTA.

FIG. 21 shows that SELTA increases expression of PD-L1 and PD-L2 in the prostate of mice in the EAP model.

FIG. 22 shows sflow cytometry data demonstrating that SELTA increases expression of PD-L1 and PD-L2 in the prostate of mice in the EAP model.

FIG. 23 shows that SELTA reduces allodynia in mice during EAP in a longitudinal study where SELTA was administered at day 21 and day 70 following induction of EAP.

FIG. 24 shows that NPI strain reducing tactile allodynia during EAP when instilled into the prostate.

FIG. 25 shows that SELTA is effective in reducing the symptoms of psoriasis in imiquimod induced psoriasis model in mice.

FIG. 26 shows that SELTA topical treatment (in water) in the imiquimod model of psoriasis reduces the epithelial thickening observed in this model.

FIG. 27 shows that SELTA is effective in reducing nociceptive pain in a model of rheumatoid arthritis (collagen induced arthritis).

FIG. 28 shows a schematic diagram of the mechanism of SELTA in reducing pain by interaction with PD-1 pathway directly on neurons.

FIGS. 29A and 29B show that SELTA and SALTA (S. aureus LTA) both reduce TLR2 expresion in dorsal root ganglia (DRG) in response to TNF-a treatment of DRG.

FIG. 30 shows that SELTA is sufficient to induce PD-1 expression in DRG.

FIG. 31 shows the empirically determined structure of SELTA.

FIG. 32 shows H-NMR data demonstrating that SELTA consists of alanylated phospho-glycerol backbone, unknown fatty acids, and potentially other yet to be identified residues

FIG. 33 shows further characterization of SELTA by ¹H and ³¹P NMR.

FIG. 34 shows ¹H, ¹³C-HSQC NMR spectra of SELTA.

FIG. 35 shows further NMR characterization of SELTA.

FIG. 36 shows NMR analysis of deacylated SELTA extract.

FIG. 37 shows that the LTA disaccharide bridging the phosphoglyceride polymer and diacylglycerol is a gentiobiose disaccharide as in SALTA. See also, Table 2.

FIG. 38 shows size exclusion chromatography of SELTA followed by NMR analysis.

FIG. 39 shows that the NMR analysis of purified deacylated SELTA comprises 45 repeating units of polyphosphoglyceride as in the structure displayed in FIG. 31.

FIG. 40 shows the fatty acid profile of the fatty acids which make up the acyl groups of the diacylglycerol moiety of SELTA. See also, Table 3.

FIG. 41 shows SELTA induced PD-L2 upregulation and CGRP downregulation in cultured dorsal root ganglion (DRG) neurons.

FIG. 42 shows increase in expression of key genes, e.g., the gene encoding prodynorphin (pdyn) and opioid receptor kappa (oprk1) in SELTA treated cells compared to control cells.

FIG. 43 shows gene expression data in SELTA treated versus control cells.

FIG. 44 shows that SELTA treatment at 21 days after initiation of experimental autoimmune prostatitis (EAP) results in a sharp reduction in tactile allodynia compared to the EAP positive control. The pain relief is eventually lost but a second treatment with SELTA produces a lasting reduction in tactile allodynia.

FIG. 45 shows Breeding strategy for creation of a sensory neuron-specific PD-1 knockout mouse.

FIG. 46 shows PD-1 expression in dorsal root ganglia (DRG) or trigeminal ganglia (TRG) in control mice (PD-1fl/f1) compared to the CKO mice (Cre^{advil}/PD-1^{f/f}).

FIG. 47 shows that wild type (WT) mice with normal PD-1 expression develop EAP with associated increase in responses indicating elevated tactile allodynia. Upon treatment with SELTA, there is a significant reduction in tactile allodynia (p=0.0279). In contrast mice with a senory neuron-specific PD-1 knockout have reduced ability to develop EAP (increased number of non-responders), develop reduced tactile allodynia in response to EAP and most importantly, are insensitive to treatment with SELTA. These data emphasize the importance of sensory neuron-specific expression of PD-1 for mediating SELTA therapeutic effects.

FIG. 48 shows that SELTA is an exogenous TLR2/6 agonist. The inventor examined SELTA receptor specificity using HEK-293 cells with expression of TLR2 along with TLR1 and TLR6, HEK293 with TLR2/TLR1 and HEK293 with TLR2/TLR6. Using NFkB transcriptional activity as the output, SELTA was shown to only activate NFkB when TLR2 was expressed in the presences of the heterodimer TLR6. This shows that SELTA is a specific exogenous TLR2/6 agonist.

### DETAILED DESCRIPTION

Disclosed herein, the inventor has shown that SELTA is a neuromodulator that activates PD-1 signaling in neurons. The inventor has demonstrated an important role for PD-1 and its natural ligands PD-L1 and PD-L2 in mediating the modulatory activity of SELTA on immune cells. PD-1 has been well characterized for reducing neuroinflammatory responses through effects on immune cell infiltrates into the CNS, particularly so in conditions where CNS damage is associated with neuroinflammation. However, accumulating evidence demonstrates that PD-1 is also expressed by CNS neurons and that PD-1 signaling can regulate neuronal excitability, synaptic transmission and plasticity via SHP-1 signaling and downstream modulation of ion channels. In the inventor's studies using SELTA, a surprising feature has been the relative rapidity of anti-nociceptive response in the inventor's experimental autoimmune prostatitis (EAP) model. The kinetics of pain alleviation suggested effects on neuronal pathways that were discrete from the immunomodulatory, disease modifying effects the inventor observed. Here, the inventor has shown that indeed following SELTA instillation, PD-1 expression is enhanced in afferent neuronal fibers in the prostate as well as primary sensory neurons at the DRG. Furthermore, PD-1 tails in these neurons were shown to be phosphorylated at tyrosine 248, a marker of inhibitory signaling. In assays examining intracellular neuronal calcium flux, SELTA pretreatment was shown to abrogate ATP-induced calcium elevation. These results lead us to hypothesize that that activation of PD-1 inhibitory signaling in neurons mediates anti-nociceptive effects of SELTA.

The inventor also has shown that SELTA is an inducer of endogenous opioid peptides/receptors in chronic pelvic pain. The inventor demonstrates that dynorphins/KORs are induced in response to SELTA and contribute to antinociceptive activity in chronic pelvic pain. Recent studies have demonstrated a role for neuronal modulation by PD-1 in opioid-induced anti-nociception and hyperalgesia.

Traditional opioids provide anti-nociception by suppressing calcium currents in dorsal root ganglia (DRG) neurons, suppressing excitatory synaptic transmission and changing membrane currents in spinal neurons, all features that have been demonstrated to be capable of being induced by PD-1. In the work disclosed here, the inventor identified, in addition to a number of genes involved in synaptic transmission and neuroinflammation, the increased mRNA expression of the Pdyn and OprK1 genes which are translated to prodynorphins and the kappa-opioid receptor (KOR) (20). In subsequent immunofluorescent analysis, the inventor observed that this increased dynorphin and KOR expression could be readily detected in the SELTA-treated DRG. These results are intriguing and differ from previous reports on PD-L1 induced PD-1 mediated anti-nociception, where the mu-opioid receptor (MOR) is involved and suggest that SELTA may use conserved mechanisms of antinociception (17), but does so in a unique manner that has implications for future therapeutic efficacy.

As used herein, Staphylococcus epidermidis lipoteichoic acid (SELTA) may refer to isolated or synthesized LTA from S. epidermidis.

### 1. SELTA May Comprise One or More Chemical Species of Formula I:

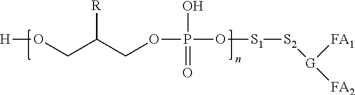

wherein n is 2-100; wherein each R is an amino acid, saccharide, or H; wherein S₁ and S₂ are monosaccharides connected by a linker; wherein G is a glyceride moiety; and wherein FA₁ and FA₂ are fatty acids.

### Neurogenic Pain

The inventor believes that, based on the mechanistic studies disclosed herein, SELTA is effective in the treatment of neurogenic pain. In contrast to nociceptive pain, neurogenic pain is a result of damage or dysfunction to the nervous system itself. Thus, one of skill in the art would not necessarily expect agents that are effective in treating nociceptive pain to be effective in the treatment of neurogenic pain. For example, neurogenic pain is typically treated with adjuvant analgesics, e.g., antidepressants and antiseizure drugs, and not with, e.g., opioids (mu opioid receptor agonists) which are effective analgesics used in the treatment of nociceptive pain. However, because the inventor has demonstrated that SELTA modifies neurons directly through PD-1 by modulating intracellular calcium concentrations in neurons as well as through the production of endogenous ligands of opioid receptor-k1, it is believed that the disclosed methods may be beneficial for the treatment of neurogenic pain.

As used herein, “neurogenic pain” refers to pain resulting from damage to or dysfunction of the peripheral or central nervous system, rather than stimulation of pain receptors. Diagnosis of neurogenic pain is suggested by pain out of proportion to tissue injury, dysesthesia (eg, burning, tingling), and signs of nerve injury detected during neurologic examination. Treatment is often with adjuvant drugs rather than analgesics (eg, antidepressants, antiseizure drugs, baclofen, topical drugs) or with nondrug treatments (eg, physical therapy, neuromodulation). A subject in need thereof may refer to a subject suffering from neurogenic pain.

Neurogenic pain may be classified according to the affected nerves: Mononeuropathies involve a single nerve, e.g., carpal tunnel syndrome, radiculopathy due to a herniated intervertebral disk. Plexopathies involve multiple nerves within a particular neural plexus; typically caused by trauma, inflammation, or nerve compression, as by a tumor.

Polyneuropathies involve multiple nerves, often throughout the body; typically caused by various metabolic disorders, paraproteinemias, toxic exposure, e.g., alcohol, chemotherapy, hereditary predisposition, or, rarely, immune mediated mechanisms.

Chemotherapy and other drugs used to treat cancer can cause peripheral neuropathy. This is termed chemotherapy-induced peripheral neuropathy (or CIPN). Certain chemotherapy drugs are more likely to cause neuropathy. These include: platinum drugs, such as oxaliplatin; taxanes, such as docetaxel; vinca alkaloids, such as vincristine; and myeloma treatments, such as bortezomib.

### Neurogenic Inflammation

Other examples of neurogenic pain include, but are not limited to, central neuropathic pain syndromes, such as resulting from multiple sclerosis, and differentiation pain, e.g., phantom limb pain.

Additional types of pain that may be treated by the disclosed methods include, but are not limited to neurogenic inflammation, migraine, complex regional pain syndrome (CRPS), rheumatoid arthritis, and vulvodynia.

As used herein, “neurogenic inflammation” refers to a form of inflammation (redness, heat, swelling, pain) initiated by activation of peripheral nervous system c-fiber neurons rather than by immunological events. The neuronal activity leads to neuropeptide release and inflammation at sites different from the original stimulus. A subject in need thereof may refer to a subject suffering from neurogenic inflammation.

Migraine: A causal association between migraine aura and headache is supported by evidence that both are linked to the phenomenon known as cortical spreading depression of Leão [3,7,8]. Cortical spreading depression is a self-propagating wave of neuronal and glial depolarization that spreads across the cerebral cortex. Cortical spreading depression is hypothesized to: Cause the aura of migraine, activate trigeminal nerve afferents, alter blood-brain barrier permeability by matrix metalloproteinase activation and upregulation.

The activation of trigeminal afferents by cortical spreading depression in turn causes inflammatory changes in the pain-sensitive meninges that generate the headache of migraine through central and peripheral reflex mechanisms. The likely molecular cascade of events by which pain-sensitive trigeminal afferent neurons are activated by cortical spreading depression involves the opening of neuronal pannexin-1 megachannels and subsequent activation of caspase-1, followed by the release of the proinflammatory mediators, activation of nuclear factor kappa-B in astrocytes, and transduction of the inflammatory signal to trigeminal nerve fibers around pial vessels [11]. Thus, this pathway links cortical spreading depression, the phenomenon thought to underlie the migraine aura, to prolonged activation of trigeminal nociception, which generates the pain of the migraine headache.

Stimulation of the trigeminal ganglion results in release of vasoactive neuropeptides, including substance P, calcitonin gene-related peptide (CGRP), and neurokinin A. Release of these neuropeptides is associated with the process of neurogenic inflammation. The two main components of this sterile inflammatory response are vasodilation (CGRP is a potent vasodilator) and plasma protein extravasation.

Neurogenic inflammation is thought to be important in the prolongation and intensification of the pain of migraine. Elevated levels of vasoactive neuropeptides have been found in the cerebrospinal fluid of patients with chronic migraine, suggesting chronic activation of the trigeminovascular system in these patients. Neurogenic inflammation may lead to the process of sensitization.

Complex regional pain syndrome (CRPS) is a disorder of a body region, usually of the distal limbs, which is characterized by pain, swelling, limited range of motion, vasomotor instability, skin changes, and patchy bone demineralization. It frequently begins following a fracture, soft tissue injury, or surgery.

The consensus definition of CRPS is as follows: “CRPS describes an array of painful conditions that are characterized by a continuing (spontaneous and/or evoked) regional pain that is seemingly disproportionate in time or degree to the usual course of any known trauma or other lesion. The pain is regional (not in a specific nerve territory or dermatome) and usually has a distal predominance of abnormal sensory, motor, sudomotor, vasomotor, and/or trophic findings. The syndrome shows variable progression over time.”

The pathogenesis of CRPS is unknown. Proposed mechanisms involve both the peripheral and central nervous system and include classic inflammation, neurogenic inflammation, and maladaptive changes in pain perception.

Proinflammatory cytokines (eg, interleukin [IL-]1beta, IL-2, IL-6, and tumor necrosis factor [TNF]-alpha) appear to have a role in CPRS. Among the mechanisms proposed for the persistent pain and allodynia that are a hallmark of CRPS is the release of inflammatory mediators and pain-producing peptides by peripheral nerves. Neuropeptides that produce pain and signs of inflammation when experimentally administered include substance P, neuropeptide Y, and calcitonin gene-related peptide. In a process known as neurogenic inflammation, nerve impulses that propagate in an antidromic fashion (that is, from proximal to distal in a sensory, particularly nociceptive, nerve axon) could lead to the release of neuropeptides. There may also be cross-talk between afferent and efferent nerves at a site of nerve injury. This model also offers a possible explanation for the phenomenon of allodynia, in which a normally painless stimulus to the affected limb, such as light touch, produces significant pain.

Central sensitization is another possible explanation for pain and allodynia in CRPS. Increased activity in nociceptive afferents due to peripheral noxious stimuli, tissue damage, or nerve injury leads to increased synaptic transmission at somatosensory neurons in the dorsal horn of the spinal cord. Cortical reorganization in sensory and motor regions of the brain and evidence of glial activation may lead to persistent activity of primary nociceptive neurons to cause central sensitization.

Rheumatoid arthritis (RA) is the most common chronic form of inflammatory arthritis, affecting approximately 1 percent of the population. It results from complex interactions between genes and environment, leading to a breakdown of immune tolerance and to synovial inflammation in a characteristic symmetric pattern. Distinct mechanisms promote and regulate inflammation and matrix destruction, including damage to bone and cartilage. Given the heterogeneous response to therapy, it is clear that RA is not just a single disease; instead, many pathways can lead to autoreactivity and synovial inflammation and result in similar clinical presentations.

The inventor's deeper understanding of the pathogenesis of RA has led to greater insights into how available therapies work and the development of new approaches to more effectively control disease activity and prevent joint injury.

Vulvodynia: Localized vulvar pain syndrome is a persistent vulvar pain that can be consistently and precisely localized to the vulvar vestibule during physical examination, has no identifiable cause, and has been present for at least three months. This syndrome has previously been referred to as vulvodynia, vestibulodynia, vulvar vestibulitis, and focal vulvitis.

Neurologic proliferation and sensitization—In women with localized vulvar pain syndrome, studies have reported increased density of intra-epithelial nerve fibers, increased vanilloid receptor VR1 (which is triggered by the inflammatory response), and hyperesthesia, which results from sensitization of the peripheral and central nervous systems. Additionally, reduced sensory and pressure-pain thresholds have been reported in patients with localized vulvar pain syndrome. Chronic inflammation of the vulvar vestibule may lead to hyperinnervation and sensitization such that even light touch to the vulva is transformed into a significantly painful response. A retrospective study of 39 patients with refractory chronic pelvic pain reported approximately two-thirds of patients demonstrated small fiber polyneuropathy, as identified by lower extremity punch biopsy. Additionally, pain can persist after the original infection or insult has cleared.

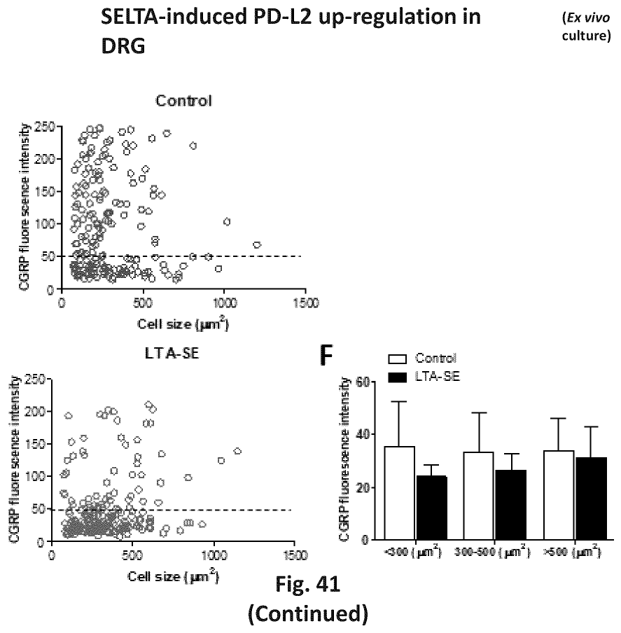

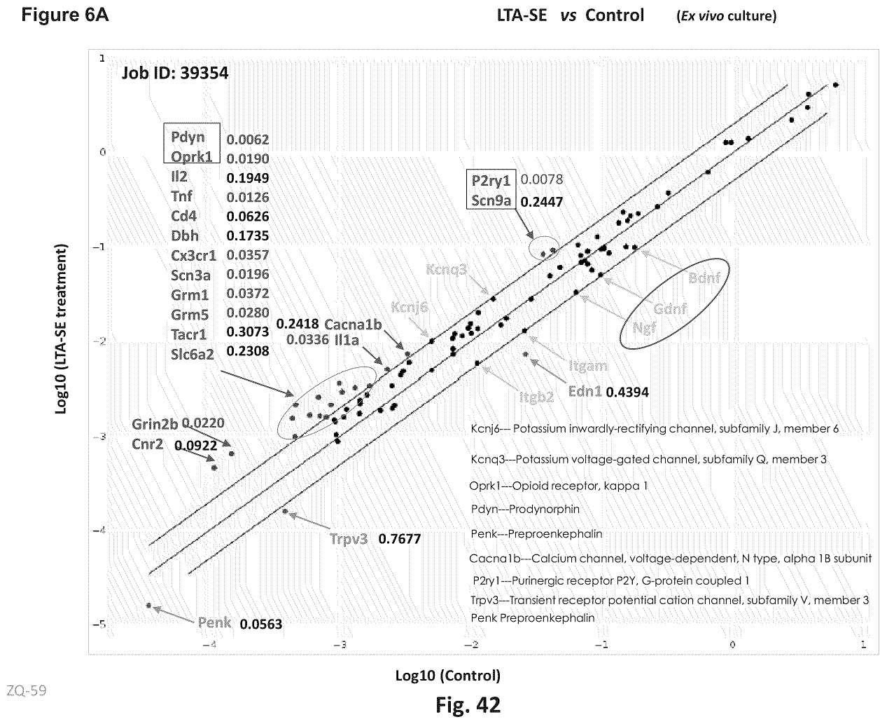

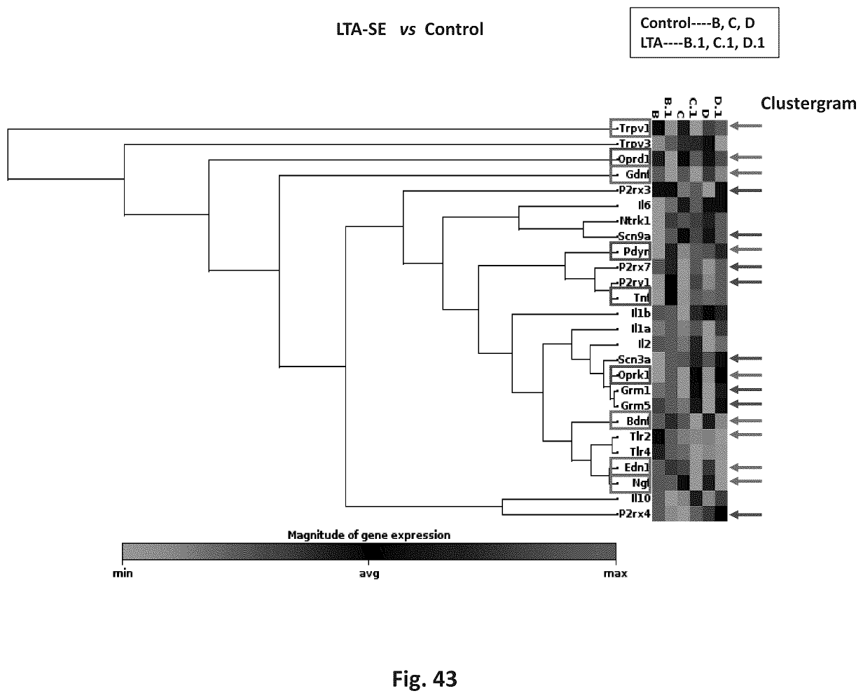

In migraines, trigeminal ganglia excitation especially through CGRP seems to be very important (hence the use of the triptans and CGRP antagonists as a treatment). The inventor has collected data demonstrating that SELTA is capable of reducing CGRP in ex vivo ganglia (FIG. 41).

As used herein, with reference to SELTA, “derived from S. epidermitis” refers to the SELTA being isolated from the S. epidermitis bacteria, as opposed to being chemically synthesized.

### Illustrative Embodiments

1. A method for treating pain in a subject in need thereof, the method comprising administering to the subject a composition comprising an effective amount of Staphylococcus epidermidis lipoteichoic acid (SELTA) for modulating the activity of the PD-1 pathway.

2. The method of embodiment 1, wherein the composition comprises live S. epidermidis bacteria.

3. The method of embodiment 1, wherein the composition comprises isolated SELTA.

4. The method of embodiment 3, wherein the isolated SELTA is derived from the cell of S. epidermidis bacteria.

5. The method of embodiment 3, wherein the isolated LTA is synthesized.

6. The method of embodiment 1, wherein the LTA comprises one or more chemical species of Formula I:

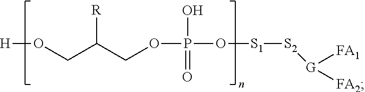

wherein n is 2-100; wherein each R is an amino acid, saccharide, or H; wherein S₁ and S₂ are monosaccharides connected by a linker; wherein G is a glyceride moiety; and wherein FA₁ and FA₂ are fatty acids.

7. The method of embodiment 6, wherein one or more R is D-alanine, a monosaccharide, or H.

8. The method of embodiment 6, wherein S₁ and S₂ are independently D-glucose, fructose, mannose, galactose, glucosamine, xylopyranose, or rhamnose, connected by linker selected from α(1→2), α(1→3), α(1→6), β(1→2), β(1→3) and (→6).

9. The method of embodiment 6, wherein FA₁ and FA₂ are independently saturated or unsaturated fatty acids.

10. The method of embodiment 1, wherein the composition is administered orally.

11. The method of any one of embodiments 1-10, wherein the subject is suffering from neurogenic pain.

12. The method of any one of embodiments 1-11, wherein the subject has been diagnosed with multiple sclerosis, phantom limb, or is currently undergoing treatment with at least one chemotherapeutic agent or has been treated with at least one chemotherapeutic agent previously.

13. The method of embodiment 12, wherein the at least one chemotherapeutic agent comprises: a taxane, a platinum containing agent, a vinca alkaloid, an epothilone, eribulin, or bortezomib.

14. The method of embodiment 13, wherein the at least one chemotherapeutic agent comprises: paclitaxel or oxaliplatin.

15. A method of modulating the expression of endogenous opioids in a subject in need thereof, the method comprising administering to the subject a composition comprising an effective amount of Staphylococcus epidermidis lipoteichoic acid (SELTA) for modulating the expression of endogenous opioids in the subject.

16. The method of embodiment 11, wherein the composition comprises live S. epidermidis bacteria.

17. The method of embodiment 11, wherein the composition comprises isolated SELTA.

18. The method of embodiment 13, wherein the isolated SELTA is derived from the cell of S. epidermidis bacteria.

19. The method of embodiment 13, wherein the isolated LTA is synthesized.

20. The method of embodiment 11, wherein the LTA comprises one or more chemical species of Formula I:

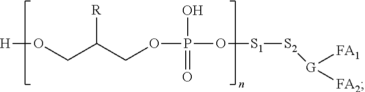

wherein n is 2-100; wherein each R is an amino acid, saccharide, or H; wherein S₁ and S₂ are monosaccharides connected by a linker; wherein G is a glyceride moiety; and wherein FA₁ and FA₂ are fatty acids.

21. The method of embodiment 16, wherein one or more R is D-alanine, a monosaccharide, or H.

22. The method of embodiment 16, wherein S₁ and S₂ are independently D-glucose, fructose, mannose, galactose, glucosamine, xylopyranose, or rhamnose, connected by linker selected from α(1→2), α(1→3), α(1→6), β(1→2), β(1→3) and β(→6).

23. The method of embodiment 16, wherein FA₁ and FA₂ are independently saturated or unsaturated fatty acids.

24. The method of embodiment 11, wherein the composition is administered orally.

25. The method of any one of embodiments 15-24, wherein the subject is suffering from neurogenic pain.

26. The method of any one of embodiments 15-25, wherein the subject has been diagnosed with multiple sclerosis, phantom limb, or is currently undergoing treatment with at least one chemotherapeutic agent or has been treated with at least one chemotherapeutic agent previously.

27. The method of embodiment 26, wherein the at least one chemotherapeutic agent comprises: a taxane, a platinum containing agent, a vinca alkaloid, an epothilone, eribulin, or bortezomib.

28. The method of embodiment 27, wherein the at least one chemotherapeutic agent comprises: paclitaxel or oxaliplatin.

29. A method of treating neurogenic pain in a subject in need thereof, the method comprising: administering a pharmaceutical composition comprising Staphylococcus epidermidis lipoteichoic acid (SELTA) to the subject to treat neurogenic pain in the subject.

30. The method of embodiment 29, wherein the subject is suffering from neurogenic pain.

31. The method of embodiment 29 or 30, wherein the subject has been diagnosed with multiple sclerosis, phantom limb, or is currently undergoing treatment with at least one chemotherapeutic agent or has been treated with at least one chemotherapeutic agent previously.

32. The method of embodiment 31, wherein the at least one chemotherapeutic agent comprises: a taxane, a platinum containing agent, a vinca alkaloid, an epothilone, eribulin, or bortezomib.

33. The method of embodiment 32, wherein the at least one chemotherapeutic agent comprises: paclitaxel or oxaliplatin.

34. A method of treating neurogenic inflammation in a subject in need thereof, the method comprising administering a pharmaceutical composition comprising Staphylococcus epidermidis lipoteichoic acid (SELTA) to the subject to treat neurogenic inflammation in the subject.

35. The method of embodiment 34, wherein the subject has been diagnosed with migraines, complex regional pain syndrome (CRPS), rheumatoid arthritis, or vulvodynia.

36. The method of embodiment 34 or 35, wherein the method reduces calcitonin related gene peptide (CRGP) in the subject.

### EXAMPLES

The following Examples are illustrative and should not be interpreted to limit the scope of the claimed subject matter.

### Example 1—SELTA Modulates Neuronal Function and Nociception

SELTA as a PD-1 mediated neuromodulator. The inventor has demonstrated an important role for PD-1 and its natural ligands PD-L1 and PD-L2 in modulatory activity of SELTA on immune cells. PD-1 has been well characterized for reducing neuroinflammatory responses through effects on immune cell infiltrates into the CNS, particularly so in conditions where CNS damage is associated with neuroinflammation. However, accumulating evidence demonstrates that PD-1 is also expressed by CNS neurons and that PD-1 signaling can regulate neuronal excitability, synaptic transmission and plasticity via SHP-1 signaling and downstream modulation of ion channels (1). In the inventor's studies using SELTA, a surprising feature has been the relative rapidity of anti-nociceptive response in the inventor's experimental autoimmune prostatitis (EAP) model. The kinetics of pain alleviation suggested effects on neuronal pathways that were discrete from the immunomodulatory, disease modifying effects the inventor observed. In preliminary studies the inventor went on to show that indeed following SELTA instillation, PD-1 expression is enhanced in afferent neuronal fibers in the prostate as well as primary sensory neurons at the dorsal root ganglion (DRG). Furthermore, PD-1 tails in these neurons were shown to be phosphorylated at tyrosine 248, a marker of inhibitory signaling (2-5). In assays examining intracellular neuronal calcium flux, SELTA pretreatment was shown to abrogate ATP-induced calcium elevation. These results lead us to hypothesize that that activation of PD-1 inhibitory signaling in neurons mediates anti-nociceptive effects of SELTA.

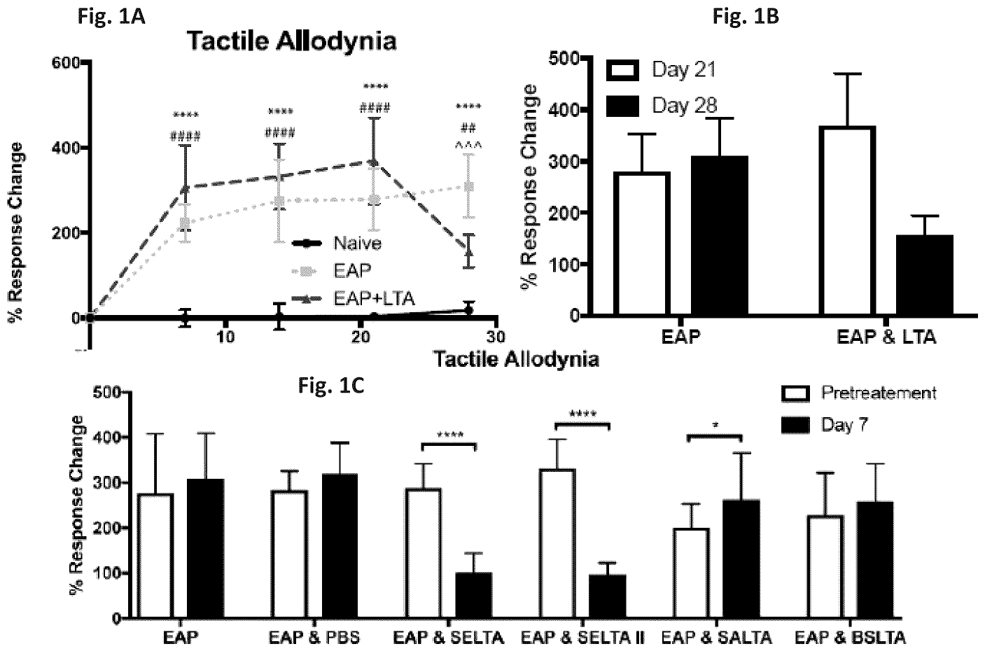

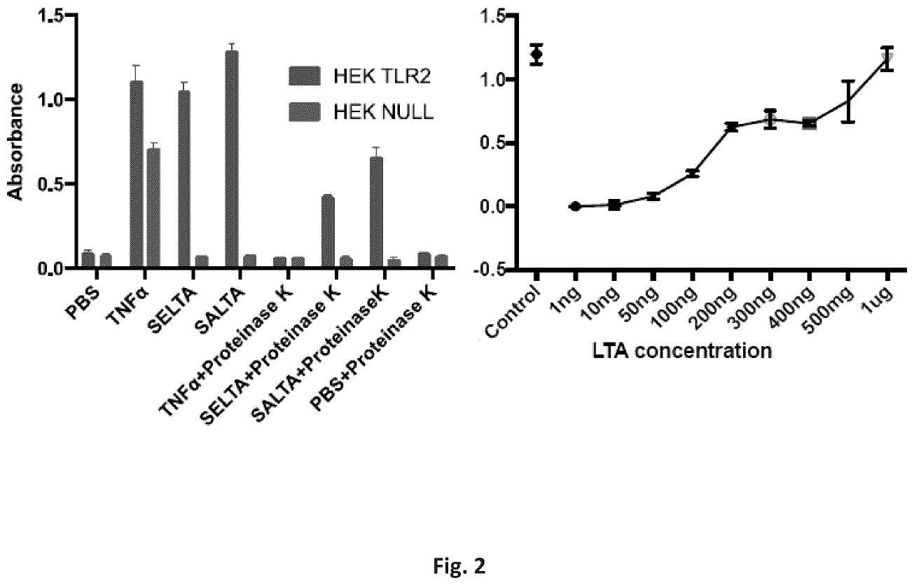

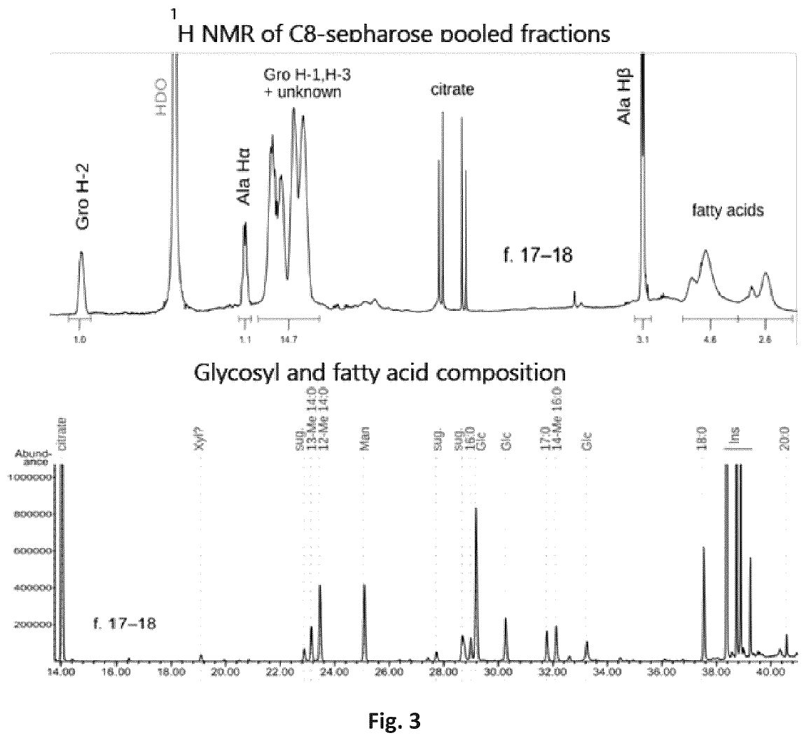

SELTA is the active principle of S. epidermidis efficacy. The strain specific nature of prostate-derived S. epidermidis to counteract Experimental autoimmune prostatitis (EAP)-induced pelvic tactile allodynia response led us to consider the exact bacterial component responsible. LTA is a major constituent of the gram-positive cell wall and studies on the immune consequences of S. epidermidis colonization have shown that LTA acts as an immunogenic factor (6, 7). To examine whether LTA was driving the observed responses in the prostates of EAP mice, the inventor isolated SELTA from the S. epidermidis strain by HPLC and intra-urethrally instilled this compound into naïve and EAP mice at day 21. In agreement with the S. epidermidis instillation data, behavioral testing revealed that SELTA treatment significantly decreased tactile allodynia responses in treated EAP mice compared to their untreated EAP-only counterparts (FIG. 1) using day 21 as a baseline. To investigate the specificity of the SELTA isolated from the S. epidermidis strain the inventor compared the ability of other LTA molecules to modulate tactile allodynia responses in EAP mice. Treatment of mice with EAP with commercially available LTA isolated from S. aureus (SALTA) or from B. subtilis (BSLTA) did not ameliorate tactile allodynia responses when compared to SELTA, after 7 days (FIG. 1).

SELTA acts via TLR2 to mediate activation of NFkB. Since LTA is known to be a ligand for TLR2, the inventor sought to confirm this for SELTA-mediated activation of NFkB. Using HEK cell lines expressing TLR2 or the null vector, the inventor showed that TLR2 expression is necessary for SELTA activation of NFkB (FIG. 2, left panel). The inventor showed that this activity could not be abolished using proteinase K, similar to LTA from S. aureus, confirming that the observed activity was not the result of protein contaminants. Using the human monocytic cell line THP-1 expressing an NFkB reporter (THP-1 blue), the inventor showed that NFkB activity in response to SELTA was increased in a dose dependent manner (FIG. 2, right panel). These results identify the target receptor for SELTA mediated effects on eukaryotic cells.

SELTA structural analysis suggests a type-1 lipoteichoic acid with a unique structure. In pilot structural characterization studies conducted with ¹H-NMR, spectra were consistent with the presence of an LTA-type polymer with characteristic glycerol, alanine and fatty acid peaks similar, but not identical with S. aureus LTA. The inventor's preliminary studies show that SELTA likely contains glycerol and alanine in fixed molar ratio, does not contain GlcNAc (unlike S. aureus), likely contains a gentiobiose fragment (like S. aureus) and has an alanylated phospho-glycerol backbone with yet-to-be-identified residue(s). In glycosyl composition analysis, only minor carbohydrate content was identified with the possibility of phosphorylation preventing detection. Several fatty acids were also detected in various proportions. These data demonstrate similarities to the basic type-1 LTA structure described for S. aureus, with important and potentially activity modifying differences.

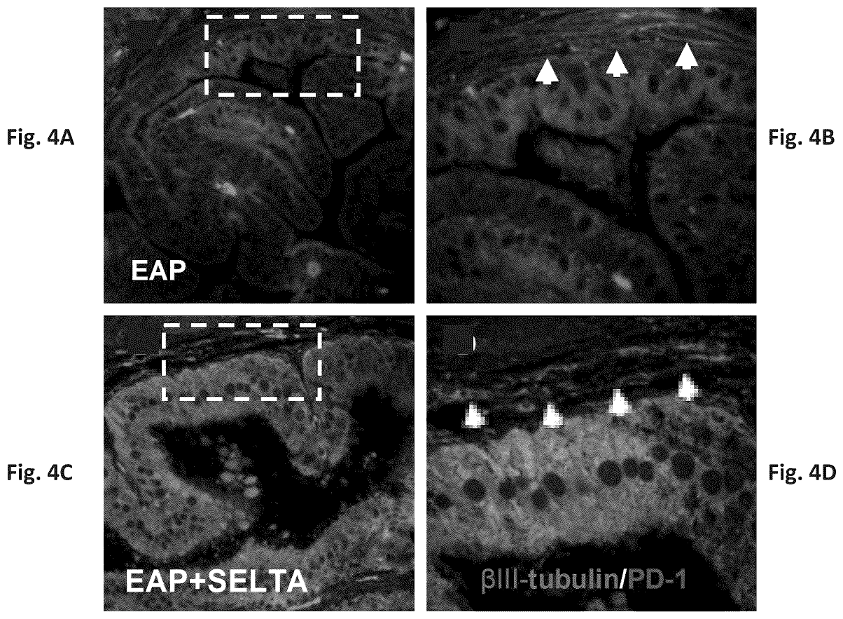

SELTA instillation induces PD-1 expression in afferent fibers of the prostate. The inventor utilized the previously established murine experimental autoimmune prostatitis (EAP) model characterized by the development of suprapubic tactile allodynia response that is chronic by day 21 (8, 9). To study whether SELTA instillation directly affects pain-associated neuronal immune checkpoint inhibitor-PD-1 expression and activation in prostate afferents, the inventor allowed the EAP model to develop till day 21 and treated the animals with an intraurethral instillation of 100 ng SELTA after measuring pre-treatment baseline pelvic pain. Animals were sacrificed at day 28. The inventor found that PD-1 immunoreactivity increased in βIII-tubulin-labelled nerve fibers in the prostate stroma (FIGS. 4B and D) of SELTA-treated EAP mice but not in the EAP control mice (FIGS. 4A and C). These data suggested association of elevated PD-1 expression following SELTA in peripheral afferent fibers of the prostate.

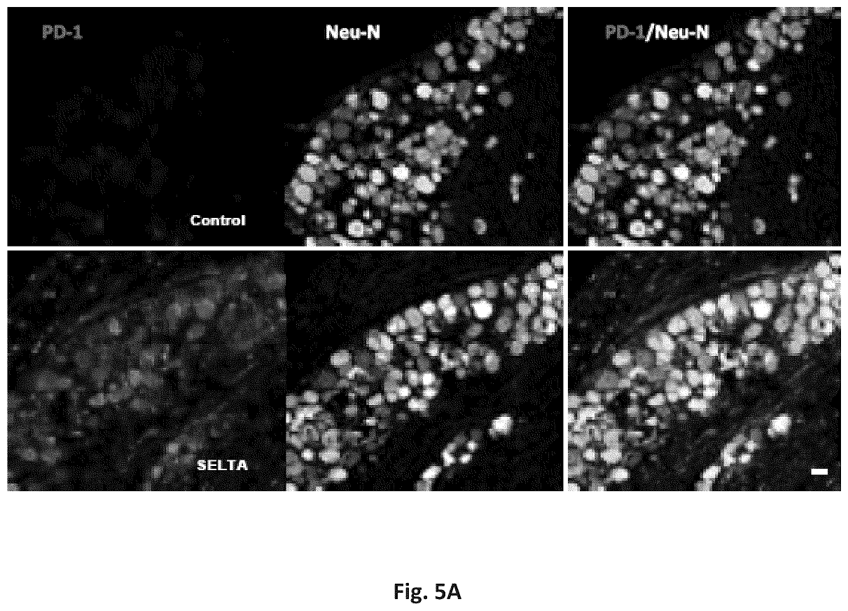

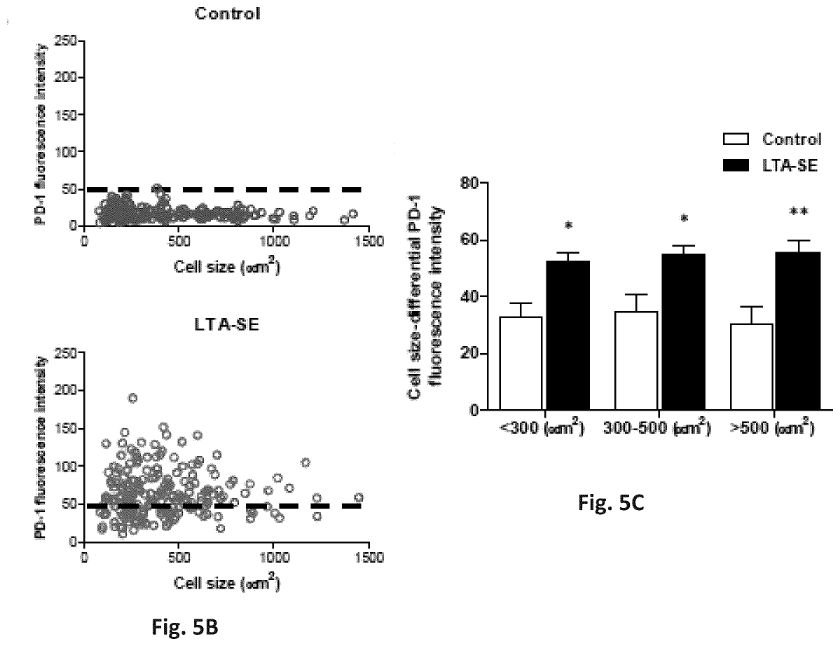

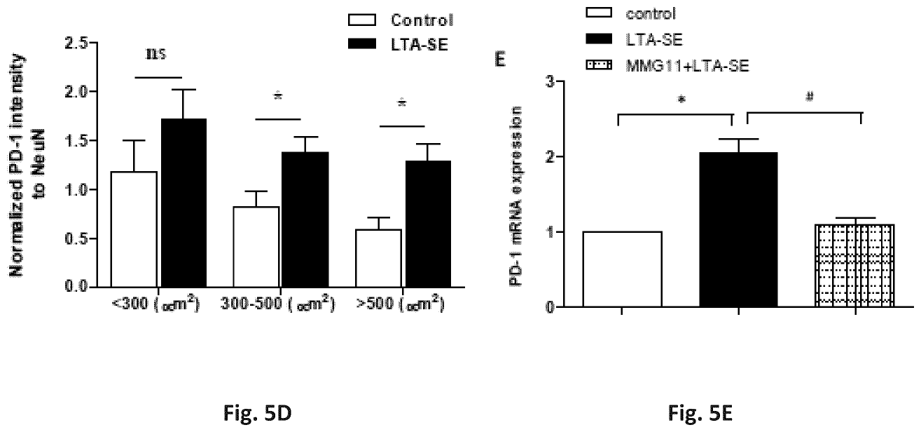

SELTA elevated PD-1 expression in primary sensory neurons. The inventor next examined PD-1 expression in the primary afferent neurons following direct exposure to SELTA. The inventor performed an ex vivo dorsal root ganglion (DRG) culture using DRG isolated at the levels of L₂ to S₁. Following 24-hour co-culture with SELTA, the expression of PD-1 was probed using immunofluorescence followed by confocal microscopy (FIG. 5A). To ascertain the cellular distribution of increased PD-1 expression, ImageJ analyses-based on individual cells were done and presented as the function of PD-1 immunoreactivity intensity to the cell size (FIG. 5B). Quantification was based on cell size-differential average fluorescence intensity (FIG. 5C). The inventor found that SELTA-treated DRG showed higher PD-1 expression regardless of the cell size. To further understand the expression of PD-1, immunoreactive intensities were normalized to the neuronal marker Neu-N (FIG. 5D). Changes were largely similar to FIG. 5C. The inventor next sought to examine whether the SELTA receptor TLR2 was involved in the modulation of PD-1 expression by SELTA. To do so the inventor utilized a specific antagonist of TLR-2, called MMG11 (10), to competitively block SELTA binding to TLR-2 and performed qPCR analysis for PD-1 mRNA expression (FIG. 5E). Blockade of TLR2 abrogated the elevation in PD-1 expression. These results strongly suggest that SELTA is capable of PD-1 induction in primary sensory neurons in a TLR2 dependent manner.

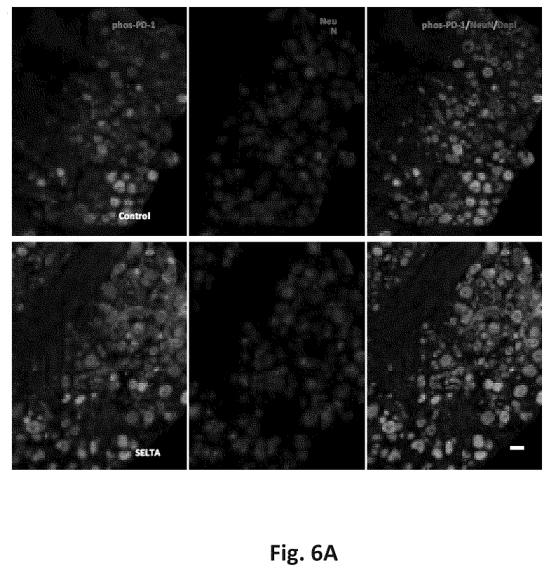

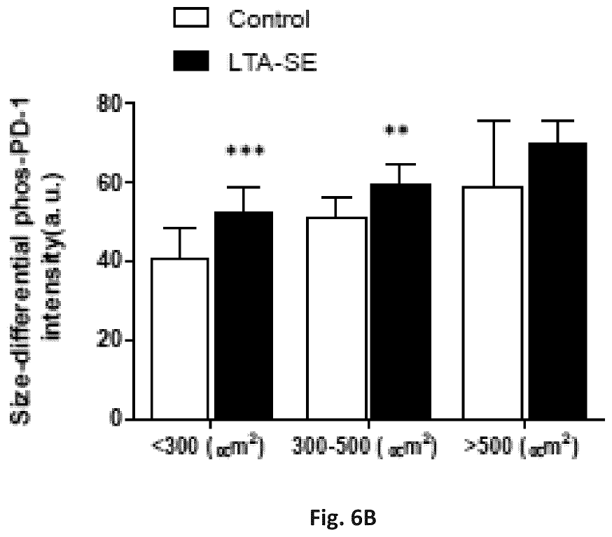

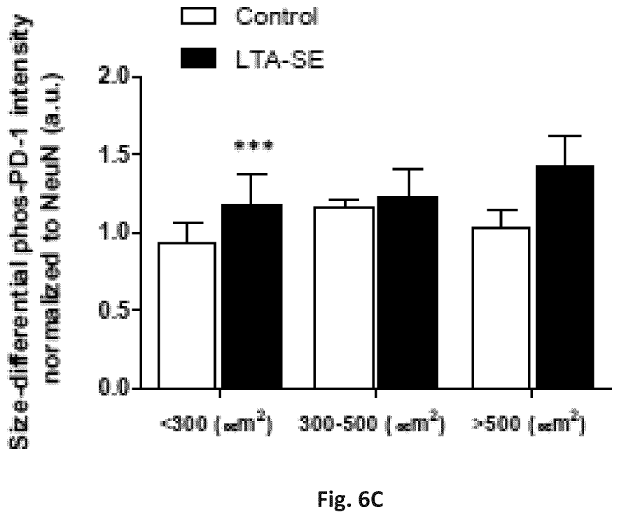

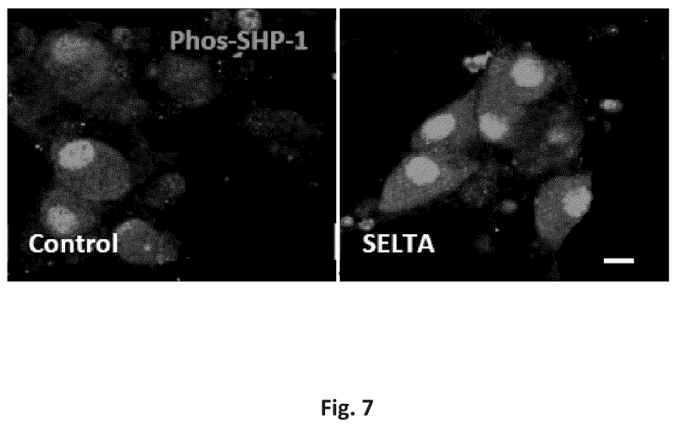

SELTA-induced inhibitory phosphorylation of PD-1 and SHP-1 in primary neurons. The phosphorylation of PD-1-Y248 mediates its immune inhibitory function in human T cells (2). To study whether SELTA is capable of activating PD-1 inhibitory signaling in DRG neurons the inventor examined the immunoreactivity of phosphorylated-PD-1-Y248. The inventor's data indicated that phosphorylated-PD-1 increased under SELTA induction (FIG. 6A-C). Furthermore, the activation of PD-1 predominantly occurred in the populations of small (<300 μm²) and medium (300-500 μm²) size neurons. Previous studies have demonstrated a role for the adapter protein SHP-1 following its phosphorylation in mediating the inhibitory signaling downstream of PD-1 in neurons (1). The inventor therefore examined phosphorylated-SHP-1 in the same trigeminal ganglia (TG) preparation (FIG. 7) and observed a significant increase in pSHP-1 following SELTA exposure. pSHP-1 immunoreactivity was increased predominantly in the nucleus as previously reported for non-hematopoietic cells (11). These results are in accordance with reports in inflammatory and neuropathic pain models (1). These results show that SELTA is capable of activating inhibitory signaling through the neuronal PD-1 receptor and involves pSHP-1.

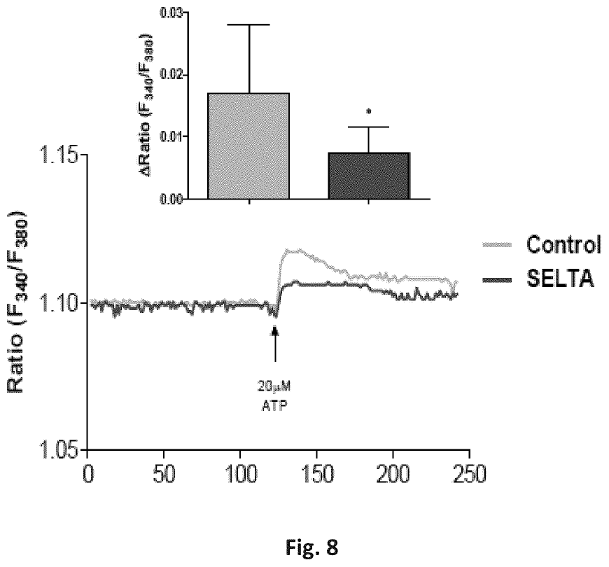

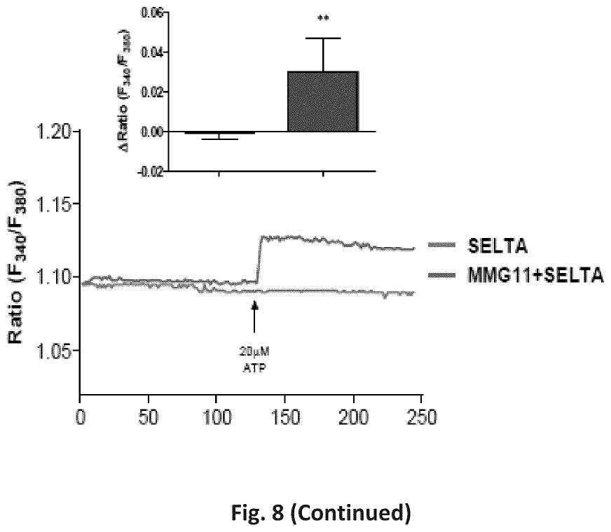

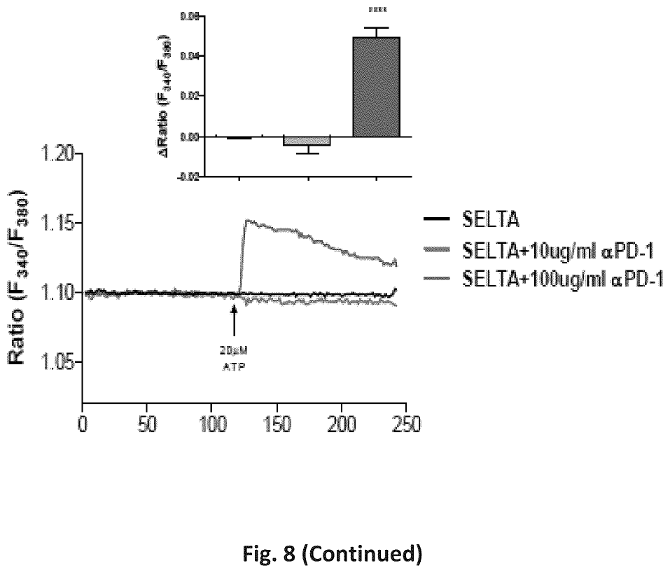

SELTA-induced inhibition of Ca²⁺ influx is reversed by neutralizing PD-1. In the transduction of nociceptive signal, Ca²⁺ influx through voltage-gated Ca²⁺ channels is critical for neurotransmitter release and synaptic transmission (12-14). To investigate whether intracellular Ca²⁺ dynamics in the afferents are affected by SELTA (8, 15), the inventor established in vitro trigeminal ganglion (TG) neuronal cultures from adult C57Bl/6 mice and performed real-time Ca²⁺ imaging using fura-2 AM (FIG. 8). Cells were pretreated with vehicle or SELTA prior to stimulation with 20 uM ATP. TLR2 specificity of these effects were examined by using MMG11, a TLR2-specific antagonist (10) and PD-1 requirement was examined by pre-incubation with an anti-PD-1 antibody (16). The inventor showed that SELTA pre-treatment significantly abrogated ATP-induced Ca²⁺ elevation (FIG. 8). Notably, preincubation of TG cultures with the TLR2 antagonist as well as a high dose of anti-PD-1 antibody abrogated the SELTA mediated inhibition of Ca²⁺ influx. These results indicate that SELTA interaction with sensory neurons can modulate neuronal Ca²⁺, and that this modulation was dependent on the expression of intact TLR2 and PD-1 signaling.

SELTA as an inducer of endogenous opioid peptides/receptors in chronic pelvic pain. The inventor demonstrate that dynorphins/KORs are induced in response to SELTA and contribute to anti-nociceptive activity in chronic pelvic pain. Recent studies have demonstrated a role for neuronal modulation by PD-1 in opioid-induced anti-nociception and hyperalgesia (17). Traditional opioids produce anti-nociception by suppressing calcium currents in DRG neurons, suppressing excitatory synaptic transmission and changing membrane currents in spinal neurons (18); all features that have been demonstrated to be capable of being induced by PD-1 neuronal signaling (19). In preliminary studies with SELTA examining ex vivo DRG using a neuroinflammatory mRNA array, the inventor identified, in addition to a number of genes involved in synaptic transmission and neuroinflammation, the increased mRNA expression of the Pdyn and OprK1 genes which are translated to prodynorphins and the kappa-opioid receptor (KOR) (20). In subsequent immunofluorescent analysis, the inventor observed that this increased dynorphin and KOR expression could be readily detected in the SELTA-treated DRG. These results are intriguing and differ from previous reports on PD-L1 induced PD-1 mediated anti-nociception, where the mu-opioid receptor (MOR) is involved and suggest that SELTA may use conserved mechanisms of antinociception (17), but does so in a unique manner that has implications for future therapeutic efficacy.

SELTA-induced differential mRNA signature in DRG. To better understand SELTA-related regulatory mechanisms and downstream signaling, the inventor's ex vivo treated DRG with 10 ng/ml SELTA or vehicle control and screened pain pathway-related gene expressions by RT Profiler PCR Array (QIAGEN, Cat. no. PAMM-162Z). A number of genes related to purinergic signaling (CX3CR1), metabotropic receptors, sodium channels (Nav1.3) and inflammation were shown to be significantly upregulated (See Table).

TABLE 1
Fold-change of SELTA and vehicle control and screened
pain pathway-related gene expression.

| Gene | Fold Regulation | p-value |
|---|---|---|
| Cx3cr1 | 4.74 | 0.03576 |
| Grin2b | 4.47 | 0.021979 |
| Grm1 | 3.8 | 0.03726 |
| Grm5 | 2.55 | 0.02801 |
| Il1a | 2.2 | 0.033601 |
| Oprk1 | 2.85 | 0.019096 |
| Pdyn | 2.86 | 0.006258 |
| P2ry1 | 2.19 | 0.007833 |
| Scn3a | 3.61 | 0.019632 |
| Tnf | 3.73 | 0.01268 |

The inventor observed a significant upregulation of mRNA for prodynorphin (Pdyn) and the opioid receptor-k1 (Oprk1) genes (Table 1, bold and italicized). These were considered to be of interest because of recent literature demonstrating that PD-L1 can mediate anti-nociceptive effects in primary sensory neurons through the mu-opioid receptor which can be acted upon by enkephalins (17). Like other opioid receptors, opioid receptor-k1 is also known to be expressed in the brain, spinal cord, and peripheral sensory neurons (20). Prodynorphin is mainly expressed in the nucleus of the neurons in the CNS and expression in the peripheral nervous system is constitutive (20).

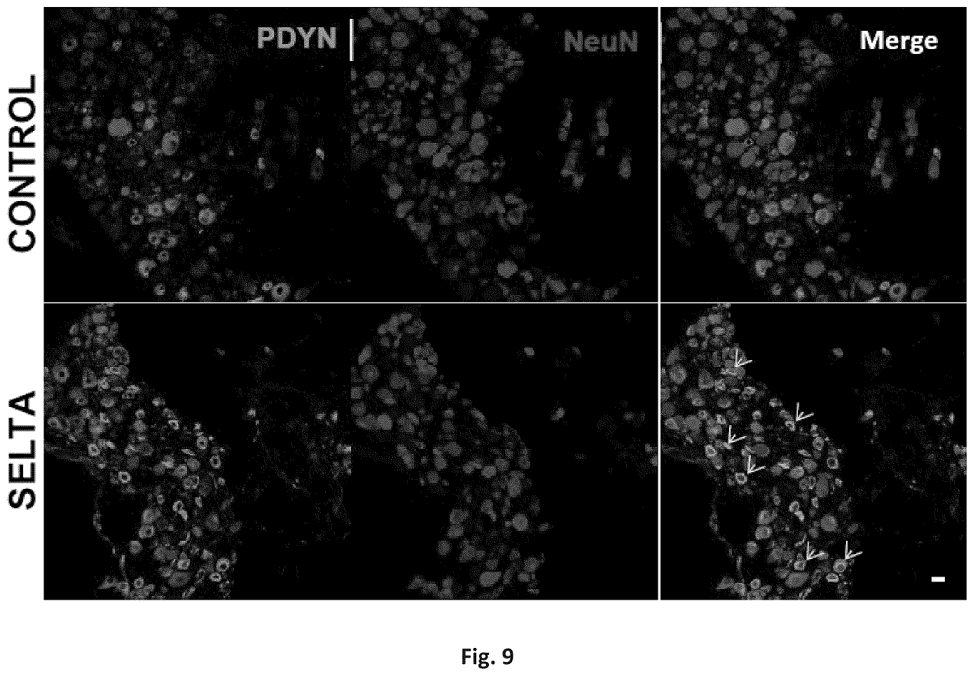

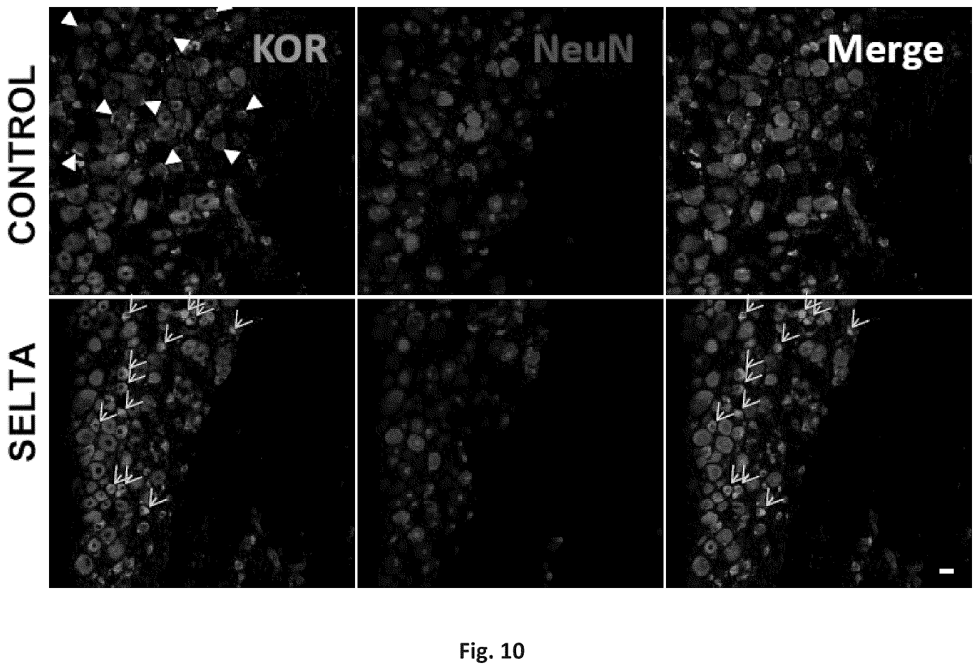

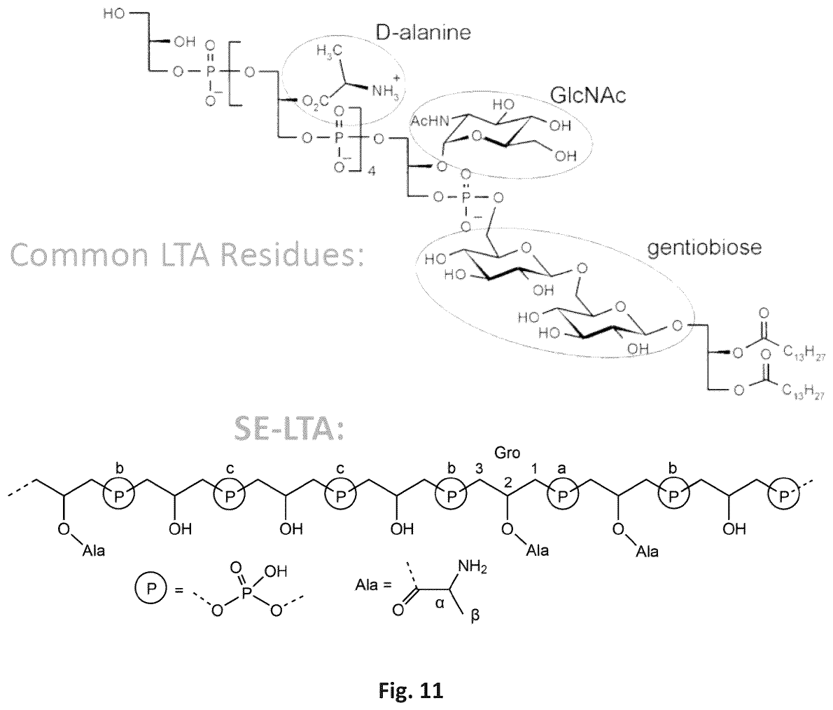

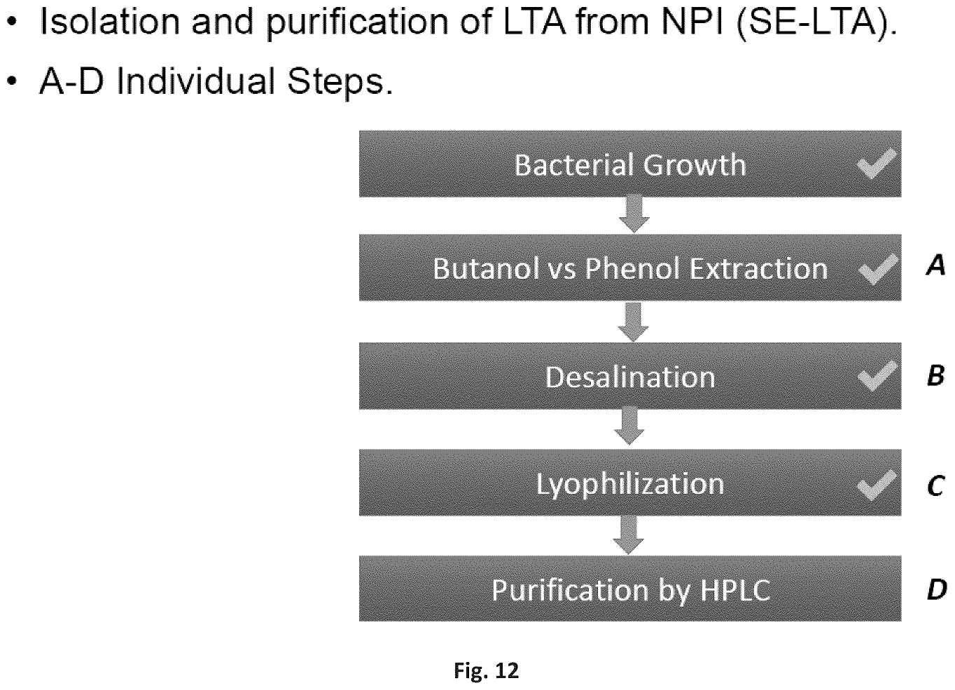

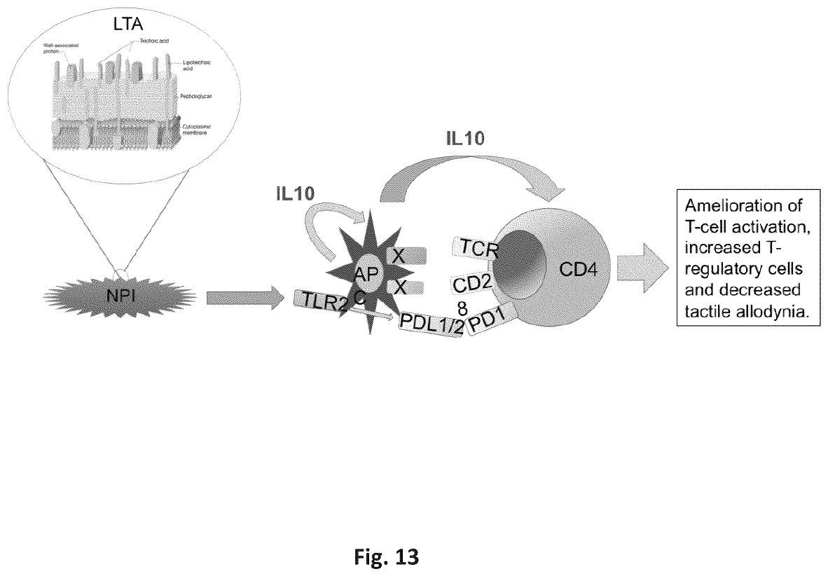

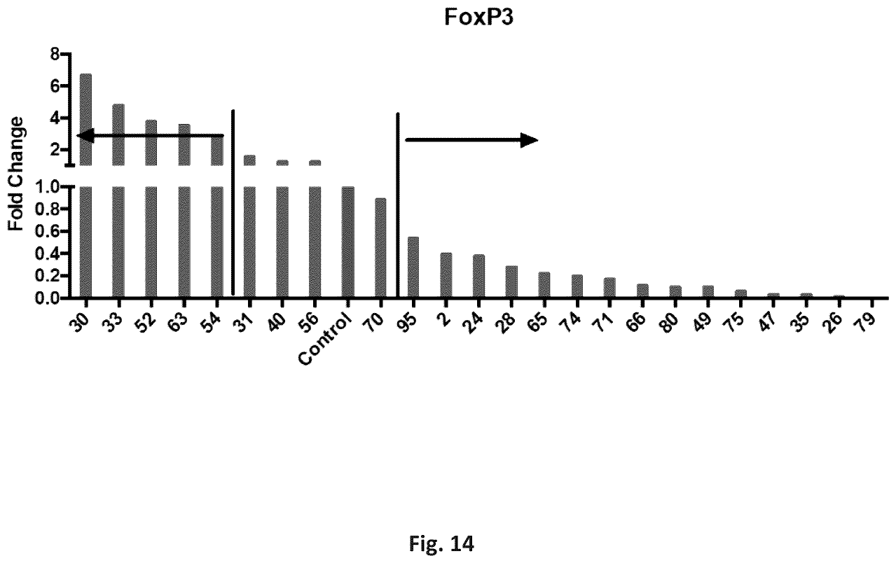

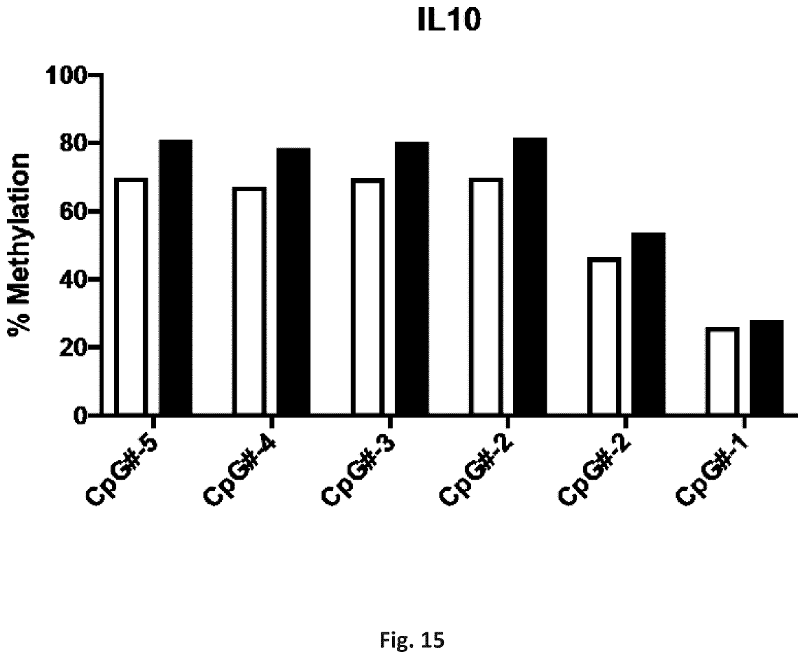

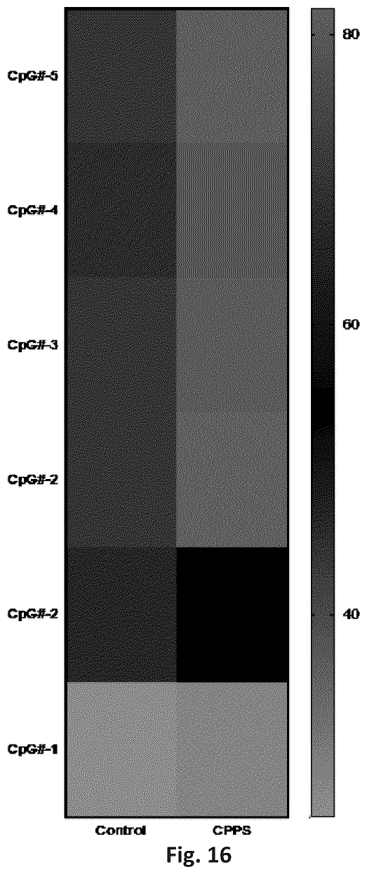

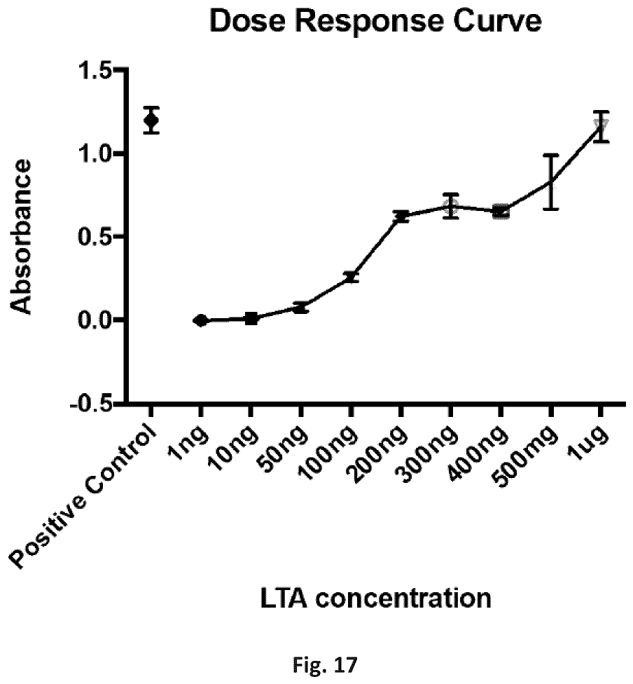

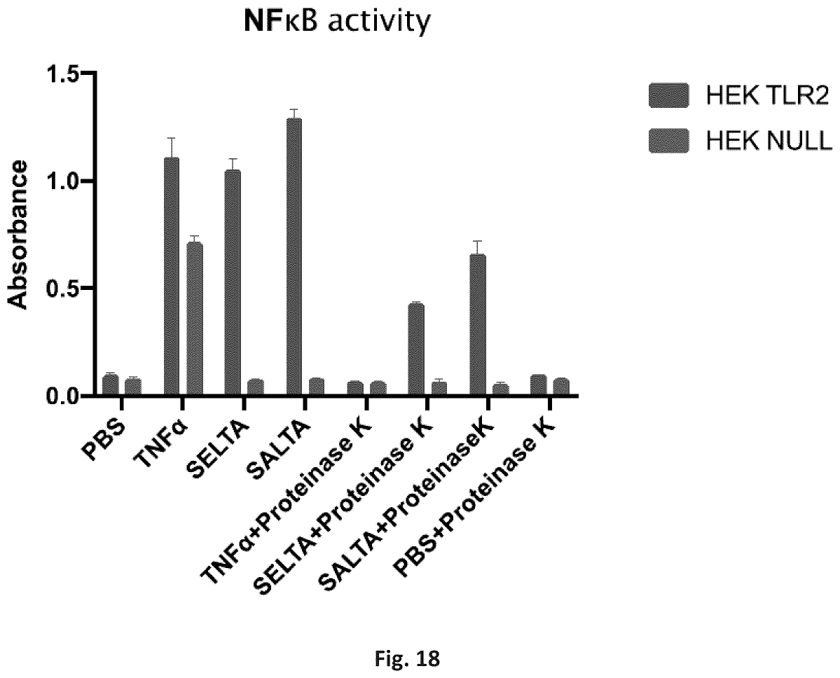

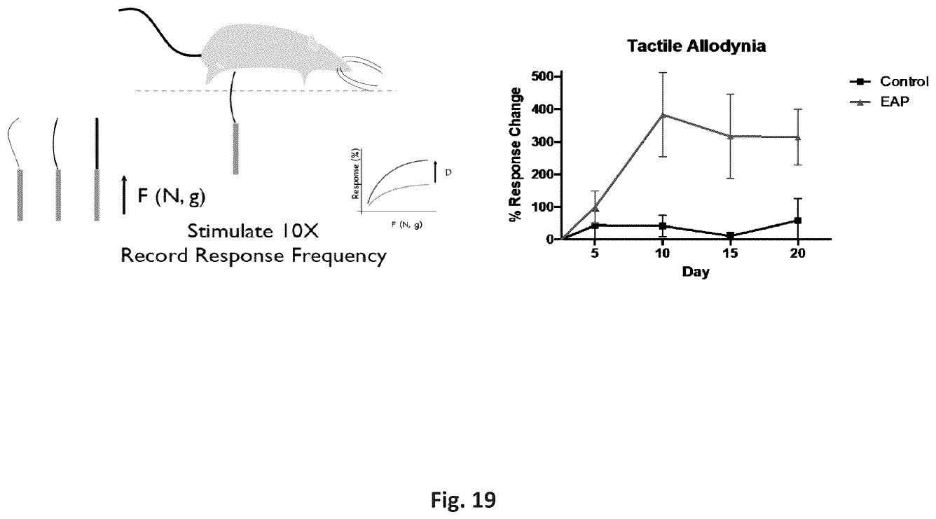

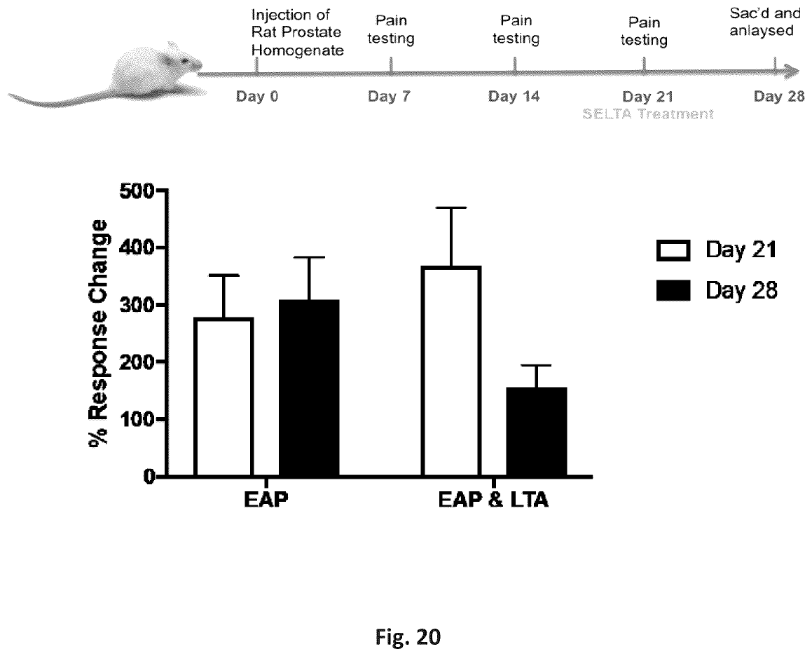

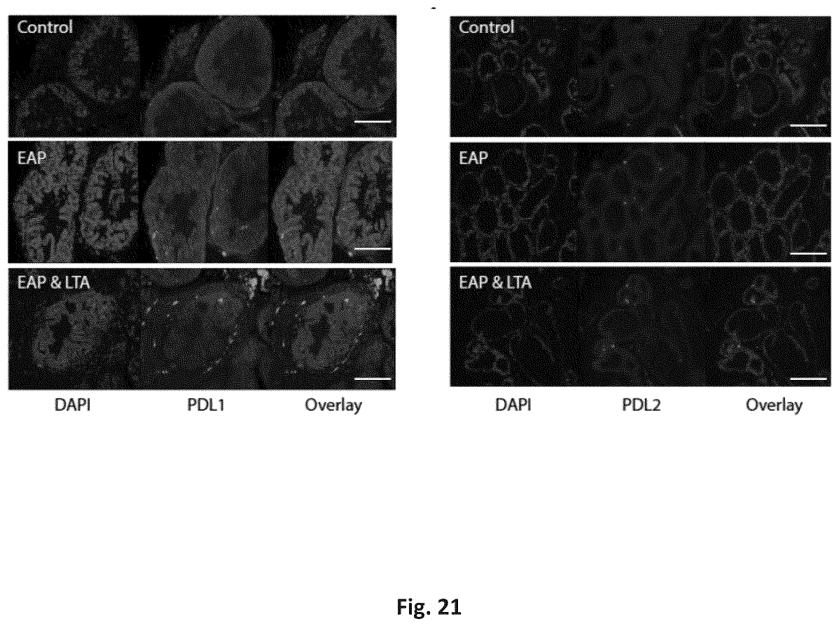

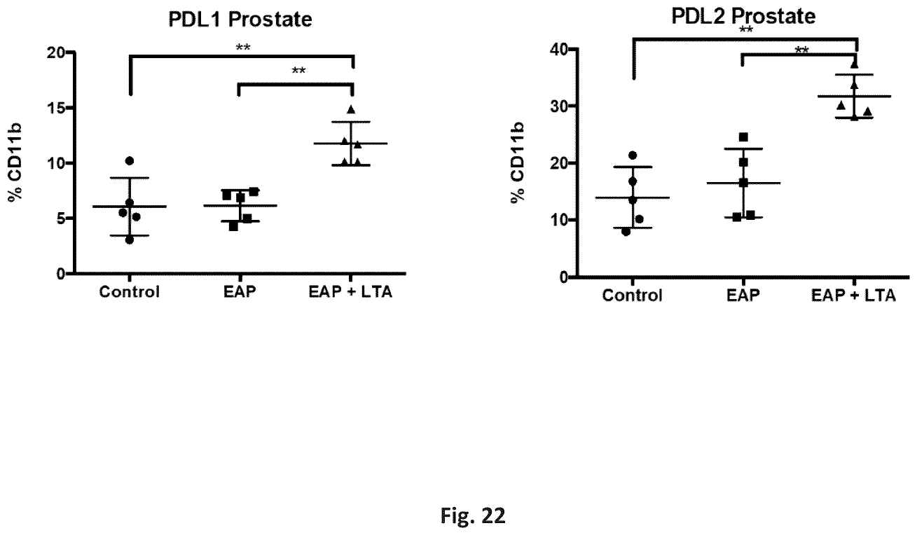

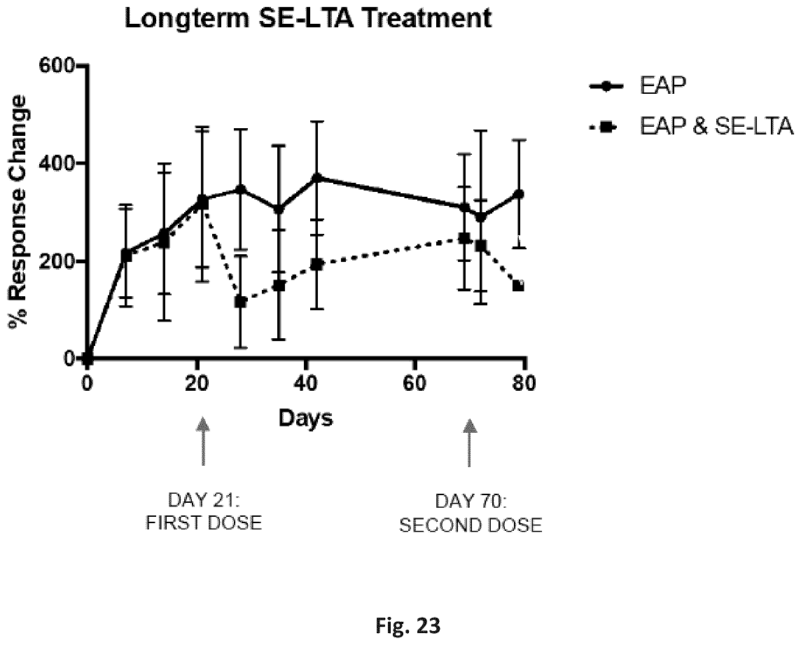

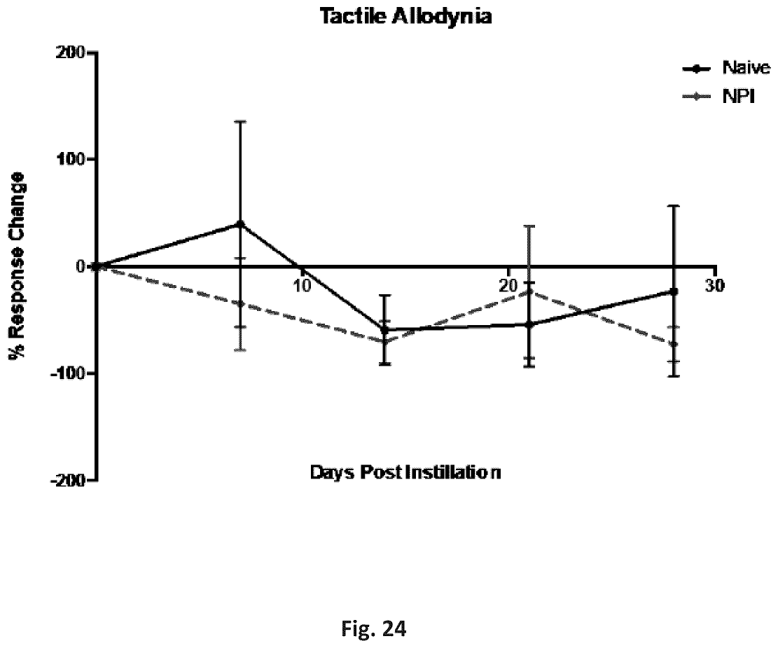

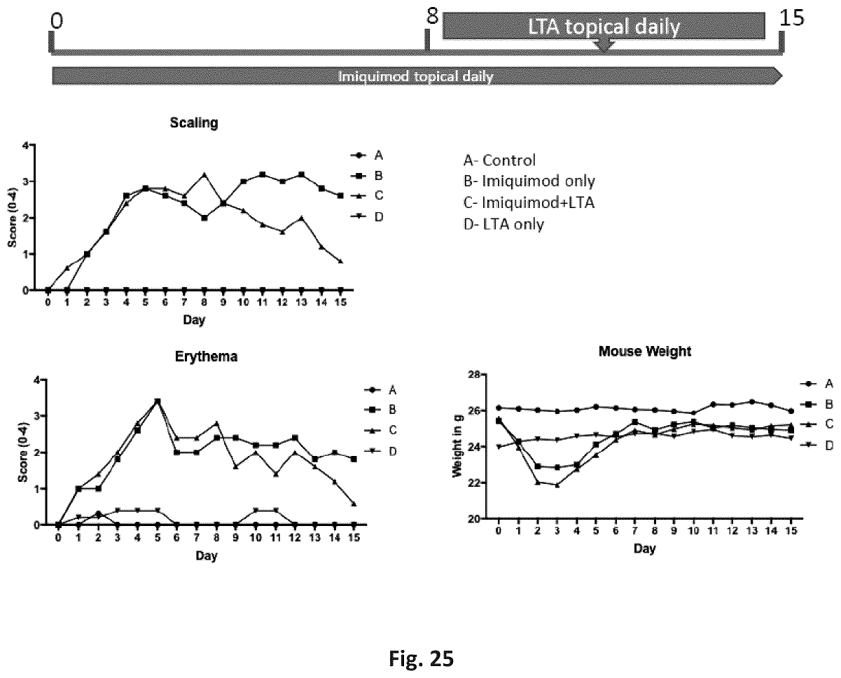

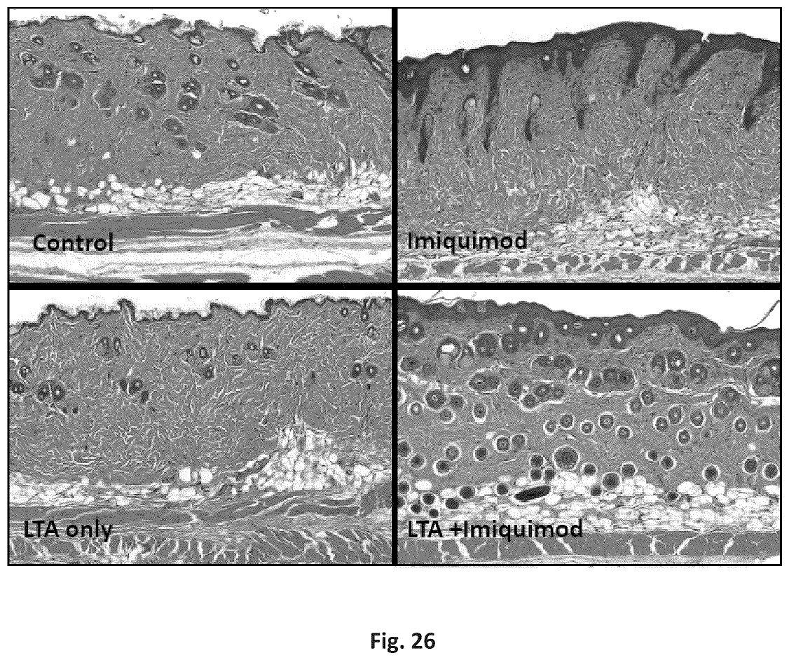

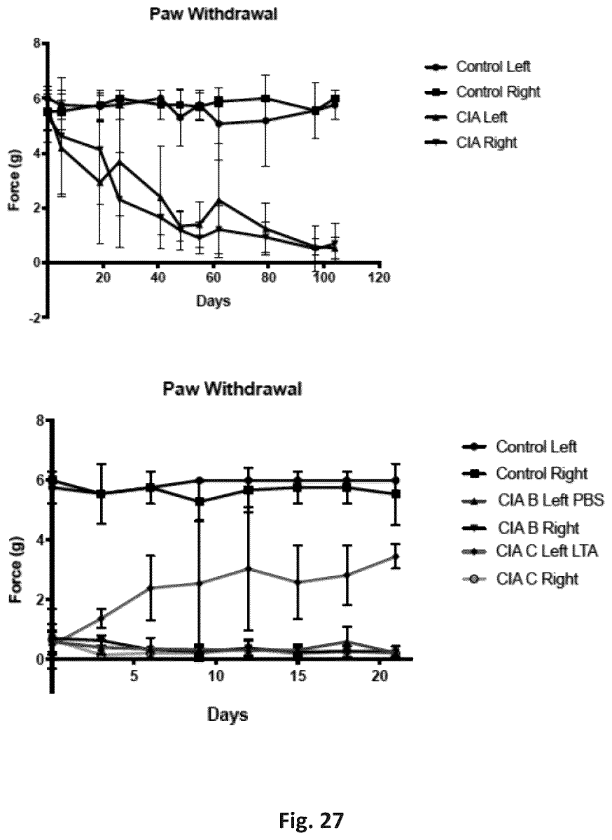

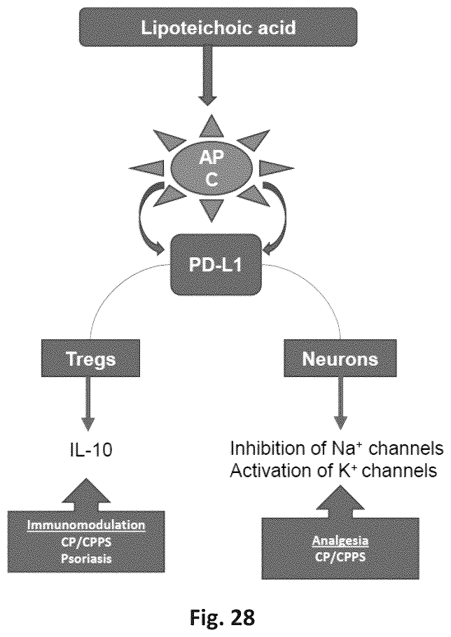

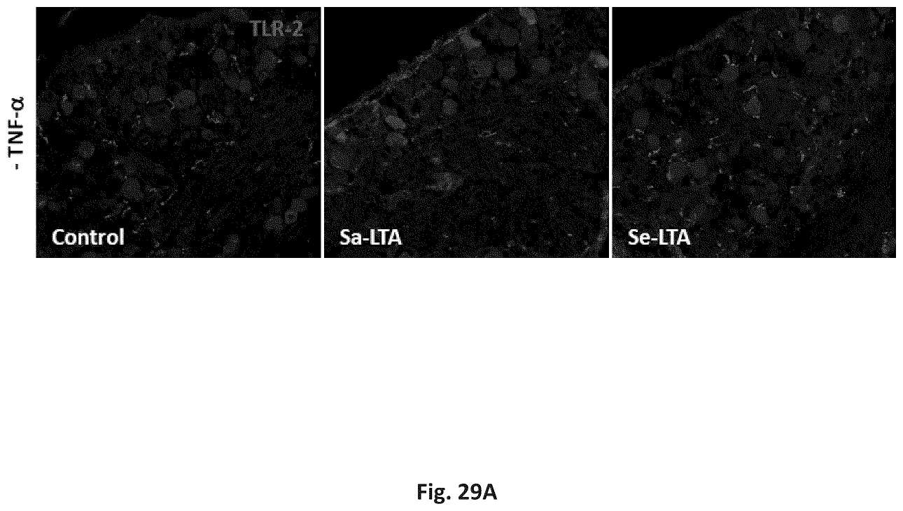

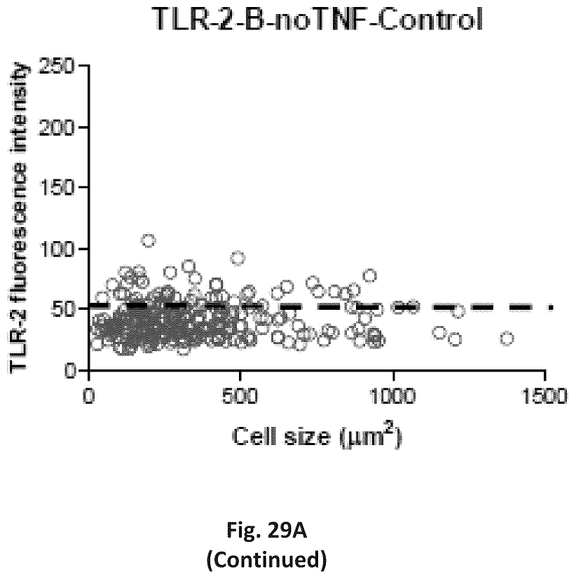

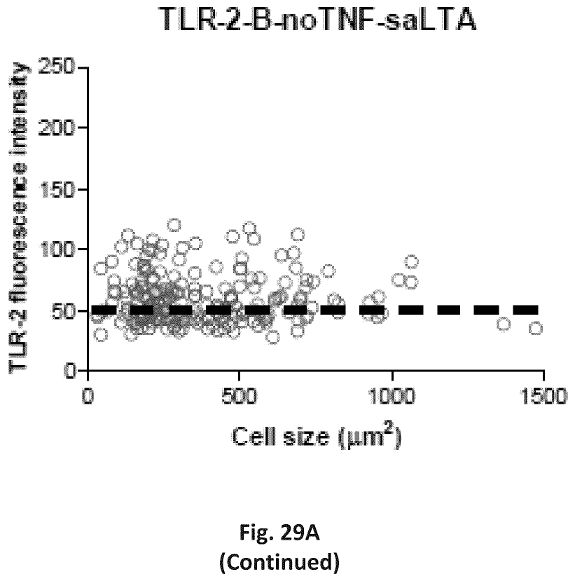

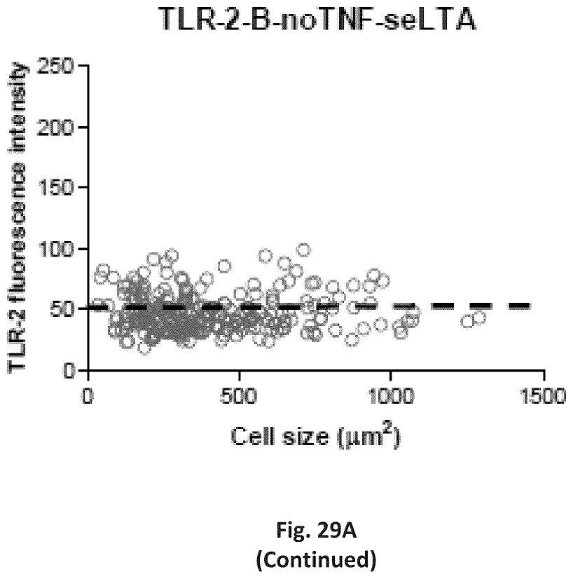

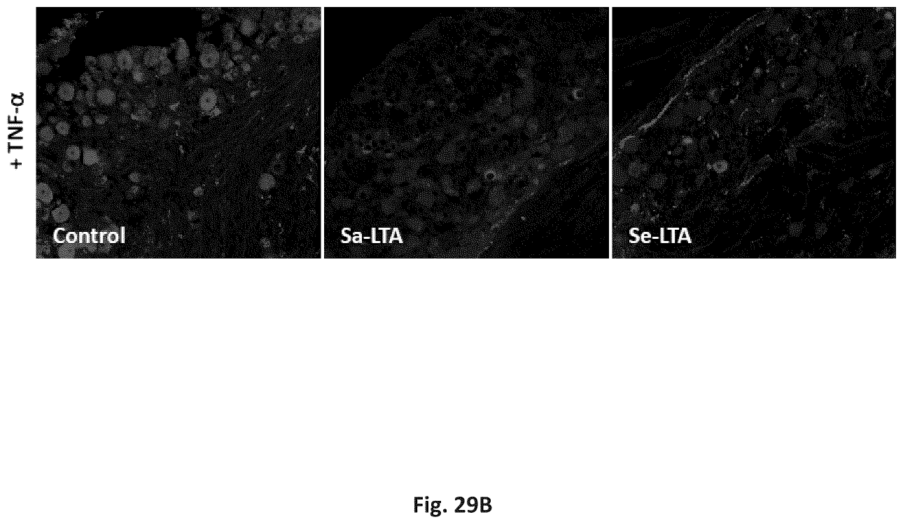

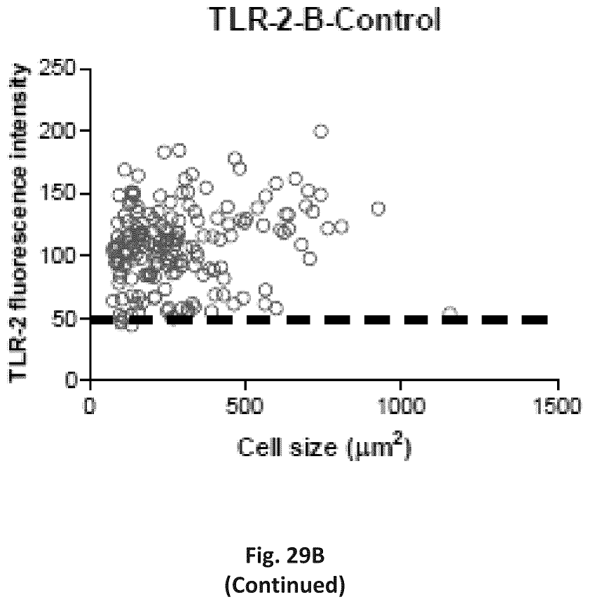

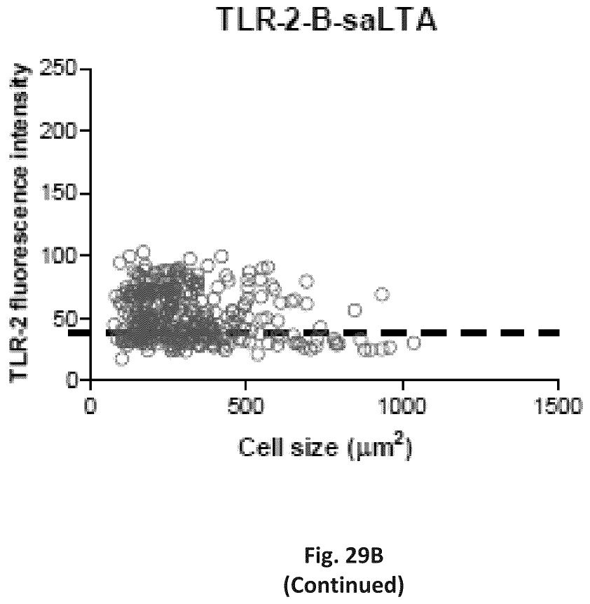

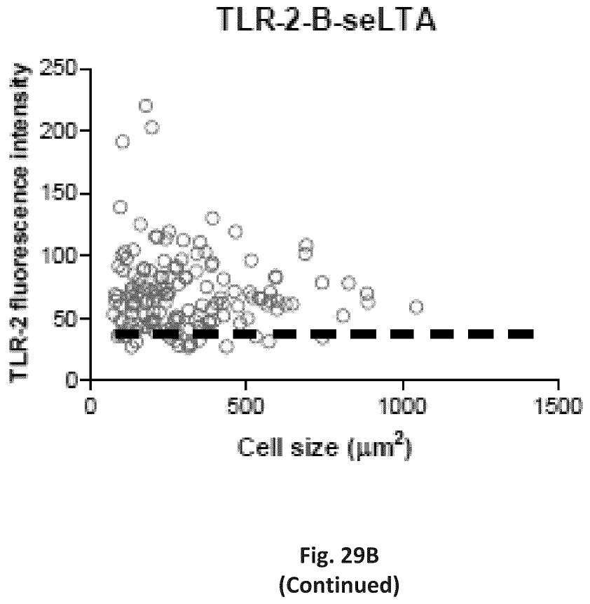

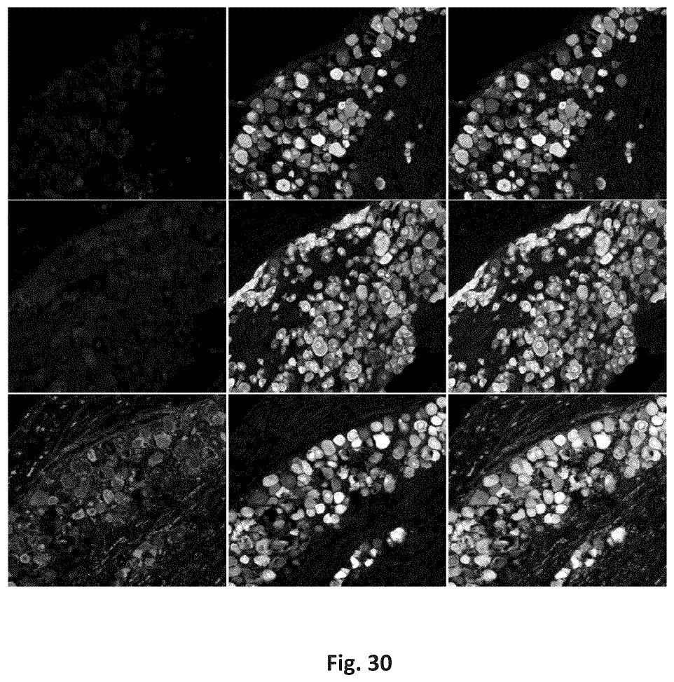

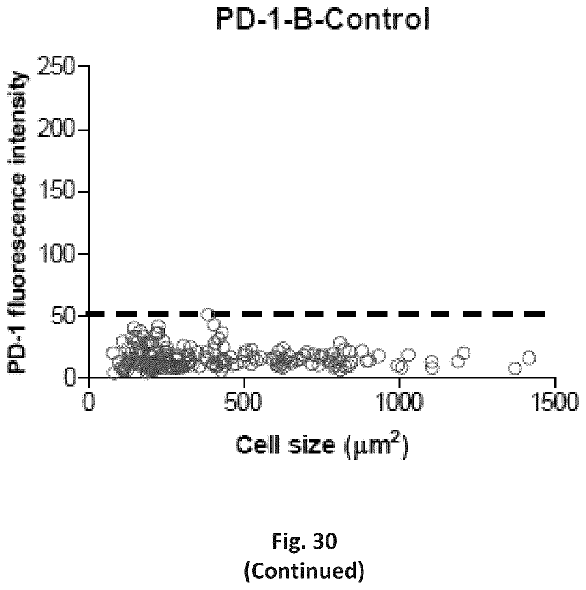

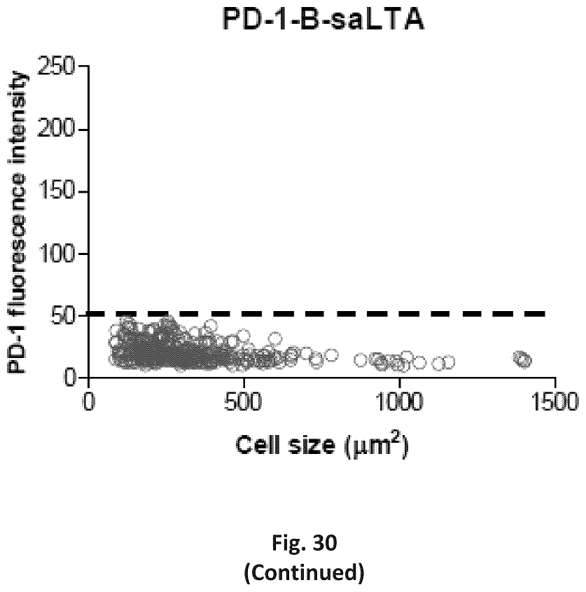

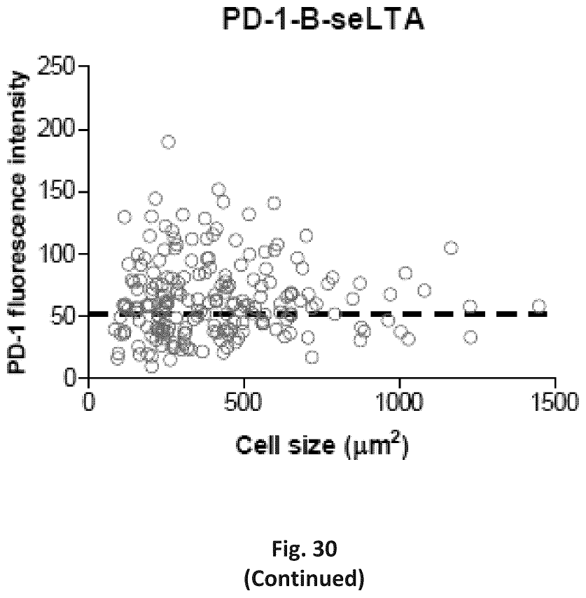

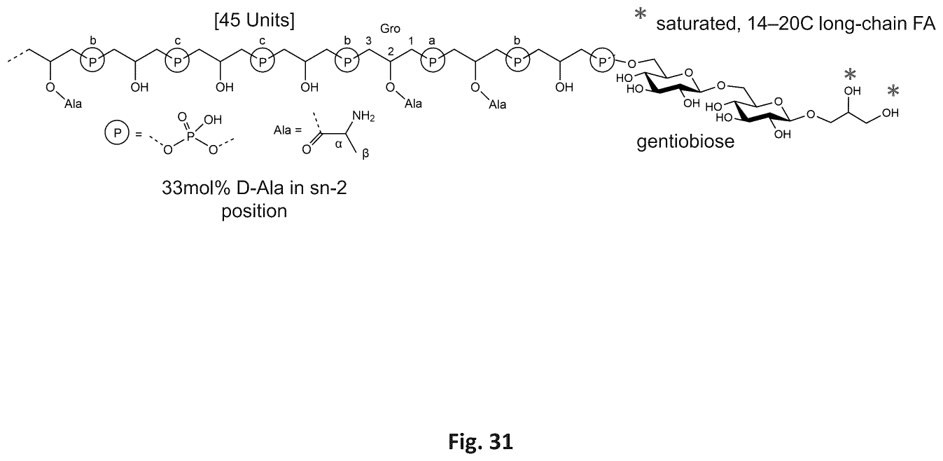

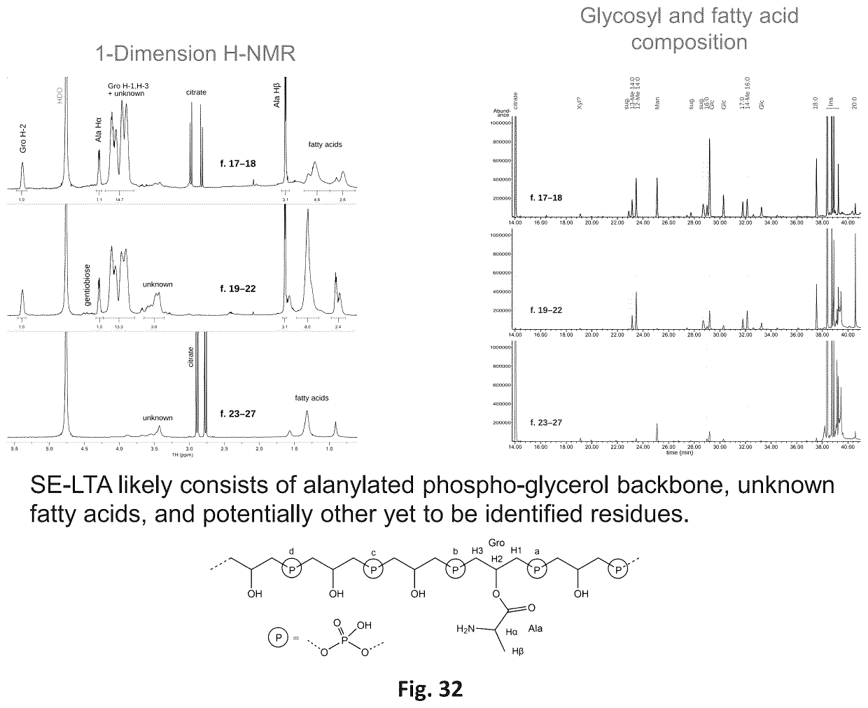

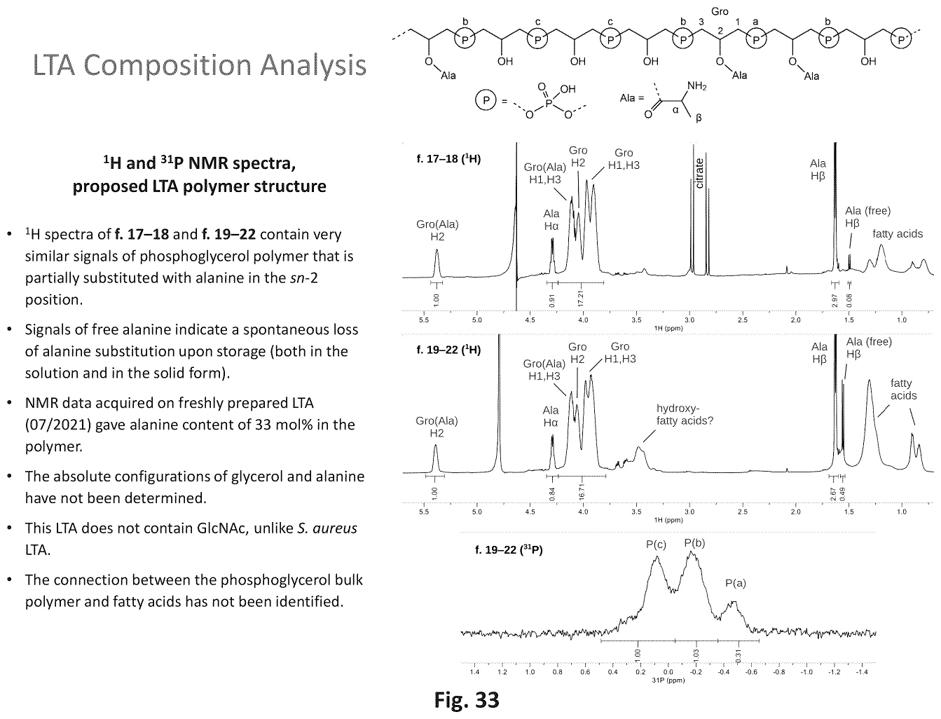

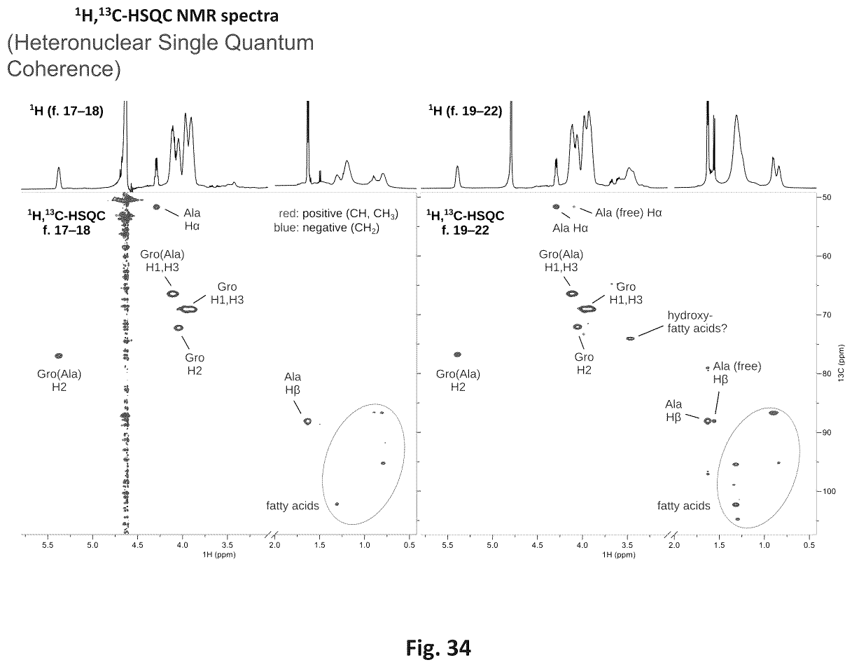

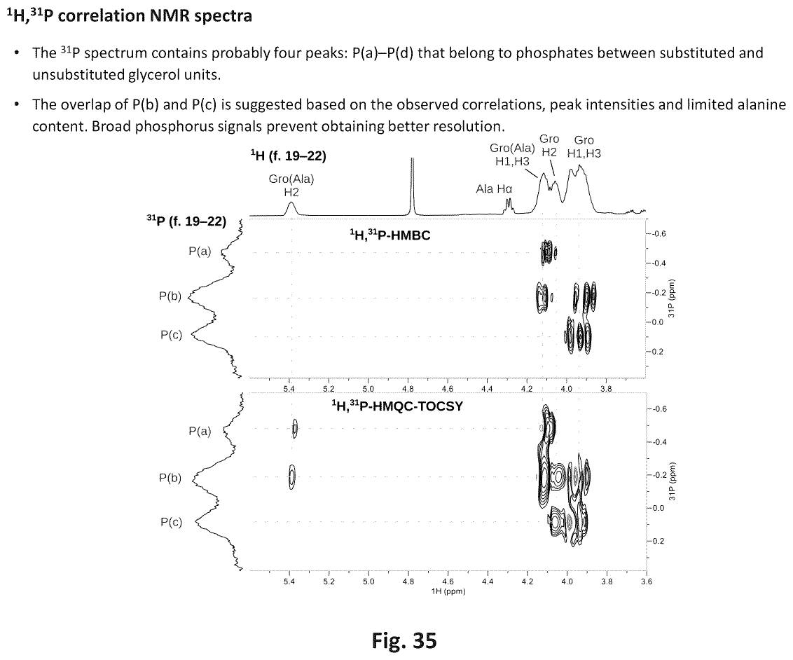

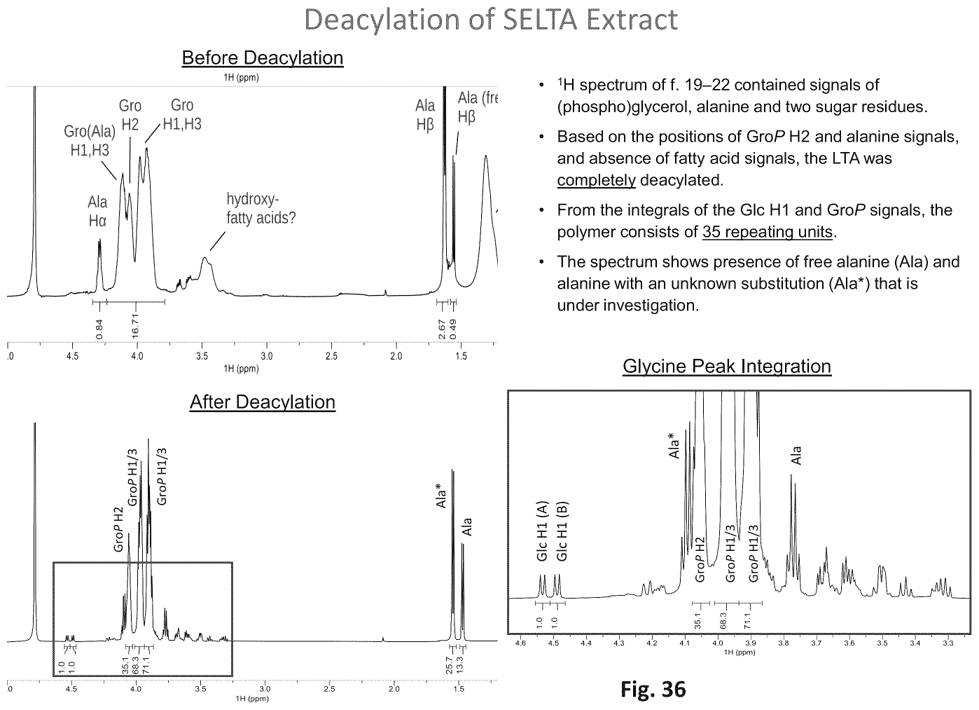

SELTA induced expression of prodynorphin and KOR in DRG. The inventor sought to confirm the mRNA expression results by examining the expression of prodynorphin and its cognate receptor-kappa opioid receptor (KOR), using immunofluorescence followed by confocal microscopy in ex vivo cultured DRG from C57BL/6J mice. DRG exposed to SELTA at 10 ng/ml showed a pronounced increase in prodynorphin immunoreactivity that was predominantly seen in the small and medium-sized afferent neurons (FIG. 9). KOR expression was also elevated but to a lesser degree in DRG neurons (FIG. 10). Here too, expression was seen to be associated with small and medium sized afferent neurons but showed a pattern of immunoreactivity where KOR expression in SELTA-treated cells was in the form of puncta in the cytoplasm compared to the cell-membrane localization in control cells. These results strongly suggest that SELTA is capable of activating endogenous opioid peptides and their cognate receptors to mediate anti-nociception.

### REFERENCES

1. G. Chen et al., PD-L1 inhibits acute and chronic pain by suppressing nociceptive neuron activity via PD-1. Nat Neurosci 20, 917-926 (2017).
2. K. Bardhan et al., Phosphorylation of PD-1-Y248 is a marker of PD-1-mediated inhibitory function in human T cells. Sci Rep 9, 17252 (2019).
3. K. Bardhan et al., Publisher Correction: Phosphorylation of PD-1-Y248 is a marker of PD-1-mediated inhibitory function in human T cells. Sci Rep 10, 15905 (2020).
4. N. Patsoukis, Q. Wang, L. Strauss, V. A. Boussiotis, Revisiting the PD-1 pathway. Sci Adv 6 (2020).
5. L. Strauss et al., Targeted deletion of PD-1 in myeloid cells induces antitumor immunity. Sci Immunol 5 (2020).
6. B. R. Huang et al., Interaction of inflammatory and anti-inflammatory responses in microglia by Staphylococcus aureus-derived lipoteichoic acid. Toxicol Appl Pharmacol 269, 43-50 (2013).
7. N. Rashidi et al., Lipopolysaccharide- and Lipoteichoic Acid-mediated Pro-inflammatory Cytokine Production and Modulation of TLR2, TLR4 and MyD88 Expression in Human Endometrial Cells. J Reprod Infertil 16, 72-81 (2015).
8. S. F. Murphy et al., Commensal bacterial modulation of the host immune response to ameliorate pain in a murine model of chronic prostatitis. Pain 158, 1517-1527 (2017).
9. Z. Liu, S. F. Murphy, L. Wong, A. J. Schaeffer, P. Thumbikat, Neuronal/astrocytic expression of chemokine (C-C motif) ligand 2 is associated with monocyte/macrophage recruitment in male chronic pelvic pain. Pain 161, 2581-2591 (2020).
10. M. Grabowski, M. S. Murgueitio, M. Bermudez, G. Wolber, G. Weindl, The novel small-molecule antagonist MMG-11 preferentially inhibits TLR2/1 signaling. Biochem Pharmacol 171, 113687 (2020).
11. G. Craggs, S. Kellie, A functional nuclear localization sequence in the C-terminal domain of SHP-1. J Biol Chem 276, 23719-23725 (2001).
12. K. Jun et al., Ablation of P/Q-type Ca2+ channel currents, altered synaptic transmission, and progressive ataxia in mice lacking the α(1A)-subunit. Proceedings of the National Academy of Sciences of the United States of America 96, 15245-15250 (1999).
13. C. Kim et al., Altered nociceptive response in mice deficient in the α1B subunit of the voltage-dependent calcium channel. Molecular and Cellular Neuroscience 18, 235-245 (2001).
14. Z. Z. Wu, S. R. Chen, H. L. Pan, Signaling mechanisms of down-regulation of voltage-activated Ca2+ channels by transient receptor potential vanilloid type 1 stimulation with olvanil in primary sensory neurons. Neuroscience 141, 407-419 (2006).
15. S. F. Murphy, C. Hall, J. D. Done, A. J. Schaeffer, P. Thumbikat, A prostate derived commensal Staphylococcus epidermidis strain prevents and ameliorates induction of chronic prostatitis by UPEC infection. Sci Rep 8, 17420 (2018).
16. J. F. Anker et al., Multi-faceted immunomodulatory and tissue-tropic clinical bacterial isolate potentiates prostate cancer immunotherapy. Nat Commun 9, 1591 (2018).
17. Z. Wang et al., Anti-PD-1 treatment impairs opioid antinociception in rodents and nonhuman primates. Sci Transl Med 12 (2020).
18. W. T. Birdsong, J. T. Williams, Recent Progress in Opioid Research from an Electrophysiological Perspective. Mol Pharmacol 98, 401-409 (2020).
19. J. Zhao, A. Roberts, Z. Wang, J. Savage, R. R. Ji, Emerging Role of PD-1 in the Central Nervous System and Brain Diseases. Neurosci Bull 10.1007/s12264-021-00683-y (2021).
20. L. D. Fricker, E. B. Margolis, I. Gomes, L. A. Devi, Five Decades of Research on Opioid Peptides: Current Knowledge and Unanswered Questions. Mol Pharmacol 98, 96-108 (2020).

### Example 2—Exemplary Protocol for the Isolation of Lipoteichoic Acid (LTA) from Staphylococcus epidermidis (SELTA)

Extraction from Cells

86.4 g cells were suspended in 432 mL [100 mM Na citrate, pH 4.7] by pipetting, 475 mL 1-butanol was added and the mixture was shaken vigorously (280 rpm) for 30 min at room temperature. The suspension was centrifuged 20 min at 13000 g and the bottom yellow water-rich phase was removed and centrifuged again (20 min @ 13000 g). The yellow phase was transferred to another bottle and centrifuged again (20 min @ 13000 g). The yellow phase (˜340 mL) was dialyzed in 1 kDa Mw cut-off bag against [2×4 L 20 mM Na citrate, pH 4.7] overnight at 4° C. and the retentate (˜400 mL) was lyophilized.

### HIC Chromatography

The dry extract (˜20 mg) was dissolved in 7 mL of [25 mM Na acetate, pH 4.5, 15 vol % 1-propanol] and centrifuged at 4000 g for 5 min to pellet insoluble material. 25 mL octyl-sepharose resin in a 16×200 mm gravity column was equilibrated with [100 mM Na citrate, pH 4.7, 15 vol % 1-propanol]=buffer A. The sample was loaded on the column by gravity at ˜0.3 mL/min, the column was washed with 90 mL buffer A (˜0.5 mL/min) and connected to an HPLC system (Agilent 1260 Infinity II). The material was eluted at 0.3 ml/min using 200 mL linear gradient from buffer A to [41 mM Na citrate, pH 4.7, 65 vol % 1-propanol], and 5 mL fractions were collected.

### Phenol-Sulfuric Acid Assay

An aliquot of 100 μL of each fraction was dried on a speedvac and dissolved in 10 μL ultrapure water. Glucose standards were prepared at 0.1, 0.3, 0.5, 0.7, 0.9 and 1.0 mg/mL. 10 μL of each sample, standard and water blank were each mixed with 200 μL concentrated H2SO4 and 40 μL 5% phenol. Following incubation at 50° C. for 30 min, absorbance at 490 nm was determined for each sample using a microplate reader.

### Total Phosphorus Assay

Selected C8-sepharose fractions and 0, 30, 60, 100, 150 and 200 nmol KH2PO4 standards were analyzed for total phosphorus content. An aliquot of 100 μL of each fraction or standard was mixed with 200 μL 10% H2SO4 in a 5 mL glass tube and heated at 200° C. for 1 hour. After cooling, 50 μL 30% H2O2 was added and the content heated at 200° C. for 40 minutes. To each cooled sample, 980 μL reagent consisting of 0.25% (w/v) Na2MoO4·2H2O and 0.5% (w/v) sodium ascorbate was added and the solution incubated at 45° C. for 20 min. Absorbance at 490 nm was determined for each sample using a microplate reader.

### NMR Spectroscopy

An aliquot of 250 μL of each selected fraction was dried on a speedvac, dissolved in 510 μL D20 (99.9% D) and transferred into a 5 mm NMR tube. NMR data were collected at 25° C. on a Bruker Avance III or Varian VNMRS spectrometer (1H, 600 MHz), each equipped with a cryogenically cooled probe. 1H data were acquired with a spectral width of 9615 Hz, 32 k complex data points and 64 transients. Prior to the Fourier transformation, NMR data were apodized with an exponentially decaying function (lb=0.3 Hz) and baselines of the spectra were corrected automatically using an 3rd-order polynomial. The spectra were referenced to citrate signal at 3.681 ppm.

### Example 3—Second Treatment with SELTA Provides Long-Lasting Reduction in Tactile Allodynia

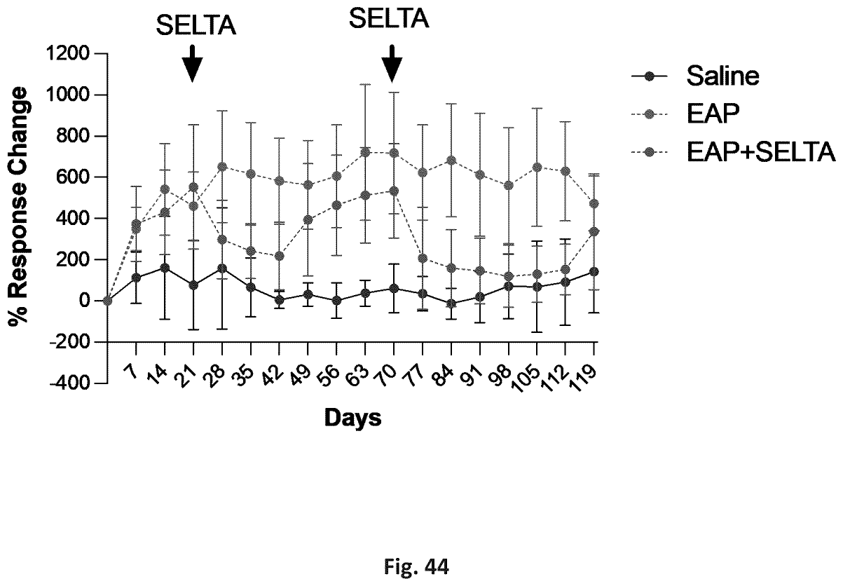

The inventor induced EAP in mice and treated the animals with SELTA at 21 days post initiation of EAP. Surprisingly, a second treatment with SELTA at day 70, resulted in a long-lasting reduction in tactile allodynia (FIG. 44).

### Example 4—Sensory Nerve Expression of PD-1 is Required for SELTA Dependent Reduction in Tactile Allodynia

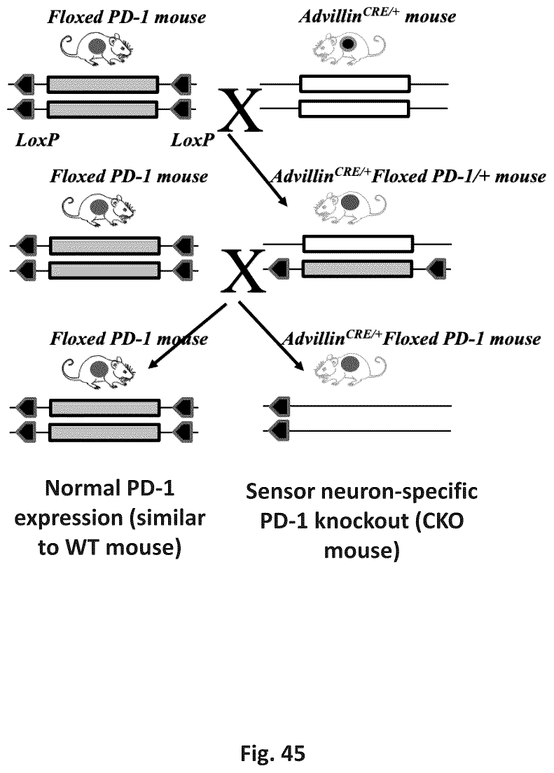

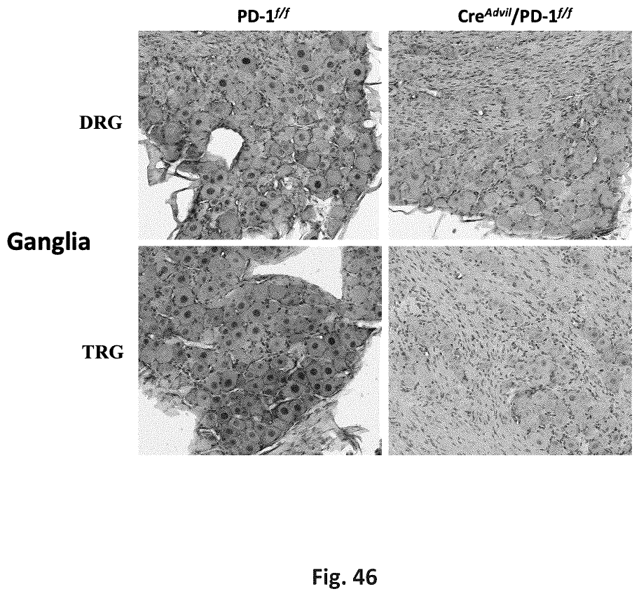

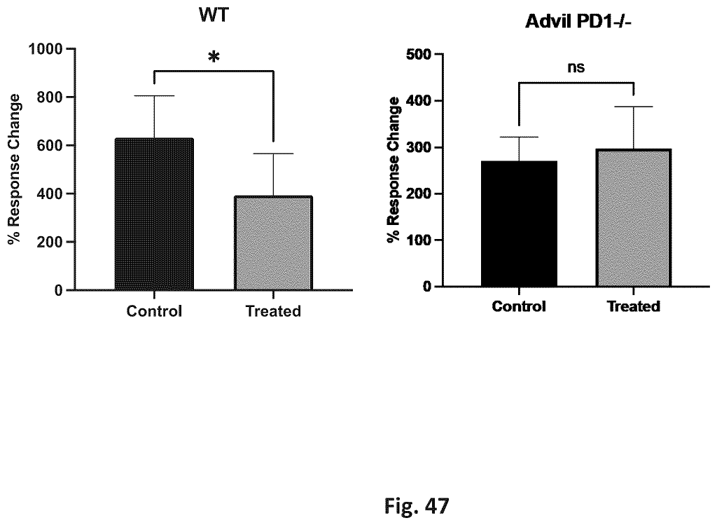

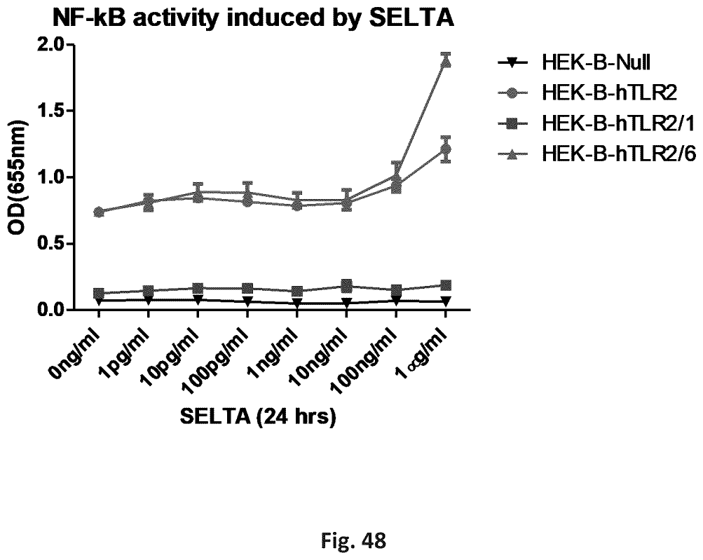

The inventor demonstrated that PD-1 expression is increased in neurons, e.g., sensory neurons, after treatment with SELTA. To test the requirement of PD-1 for the observed SELTA-dependent reduction in tactile allodynia, the inventor created a conditional PD-1 knockout in advillin-expressing sensory nerves (FIG. 45). The inventor demonstrated that PD-1 expression was lost in dorsal root ganglia (DRG) and trigeminal root ganglia (TRG) in conditional knockout animals (Cre^{advil}/PD-1^{f/f}) as compared to control samples (PD-1^{f/f}) (FIG. 46).

The inventor induced EAP in WT and conditional knockout (Advil PD1−/−) animals and demonstrated that PD-1 expression in sensory nerves is required for the observed SELTA dependent reduction in tactile allodynia as WT, but not PD-1 conditional knockout animals, demonstrated a significant reduction in tactile allodynia after treatment with SELTA.

The inventor further examined SELTA receptor specificity using HEK-293 cells with expression of TLR2 along with TLR1 and TLR6, HEK293 with TLR2/TLR1, and HEK293 with TLR2/TLR6. Using NFkB transcriptional activity as the output, SELTA was shown to only activate NFkB when TLR2 was expressed in the presence of the heterodimer TLR6. This shows that SELTA is a specific exogenous TLR2/6 agonist.

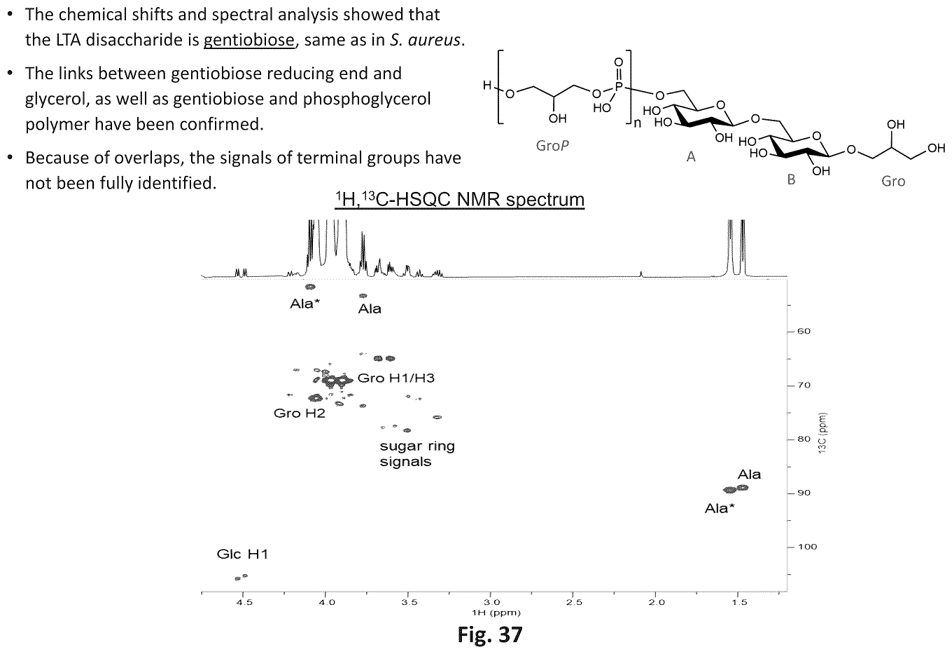

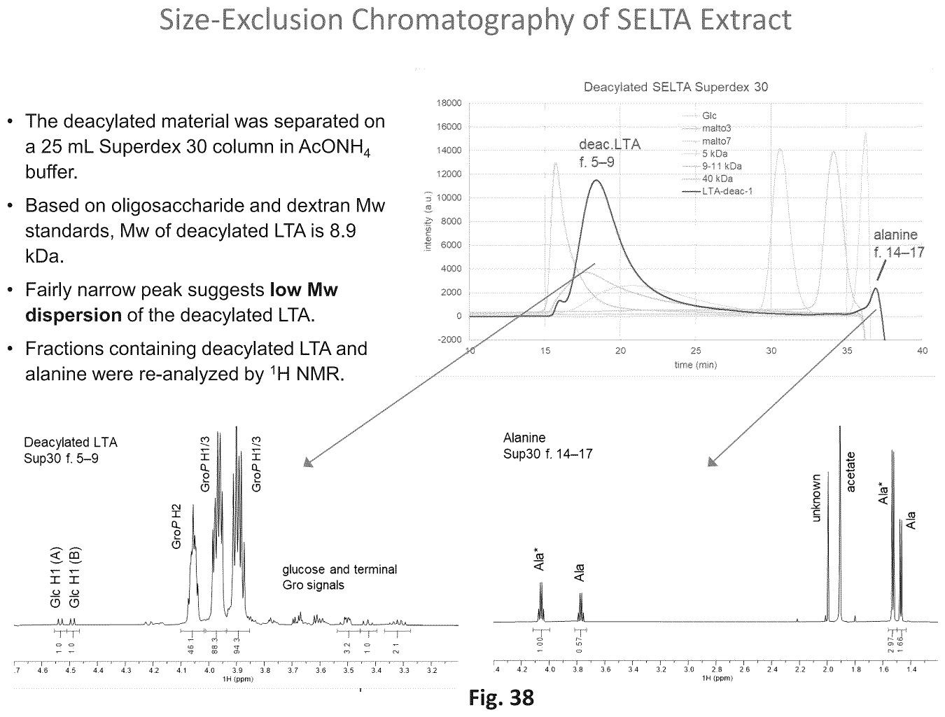

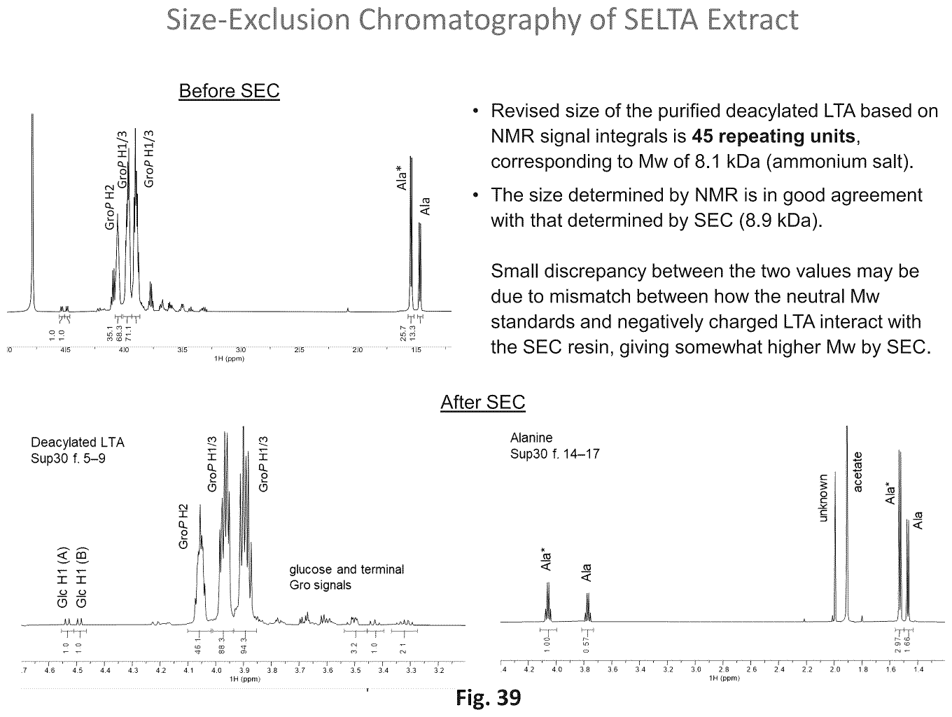

TABLE 2
¹H, ¹³C-HSQC NMR spectrum. See also, FIG. 37.

| | | Group | | | | | |
|---|---|---|---|---|---|---|---|
| Residue | | 1 | 2 | 3 | 4 | 5 | 6(a, b) |
| A | 1H | 4.53 | 3.33 | 3.50 | 3.50 | 3.58 | 4.18, 4.05 |
| P-bGlc(1-6) | 13C | 105.7 | 75.8 | 78.3 | 71.9 | 77.4 | 67.0 |
| | 31P | | | | | | 0.80 |
| B | 1H | 4.49 | 3.31 | 3.51 | 3.43 | 3.65 | 4.21, 3.85 |
| bGlc(1- | 130 | 105.2 | 75.8 | 78.2 | 72.4 | 77.7 | 71.6 |
| C | 1H | 3.97/3.90 | 4.06 | 3.97/3.90 | | | |
| GroP | 13C | 68.9 | 72.2 | 68.9 | | | |
| | 31P | 0.77 | | | | | |
| D | 1H | | | | | | |
| t-GroP | 13C | | | Links | | | |
| | | | | A6 - GroP | | | |
| | | | | A1 - B6 | | | |
| | | | | B1 - E1 | | | |
| | 31P | | | | | | |
| E | 1H | 3.77 | | | | | |
| Gro | 13C | 73.6 | | | | | |

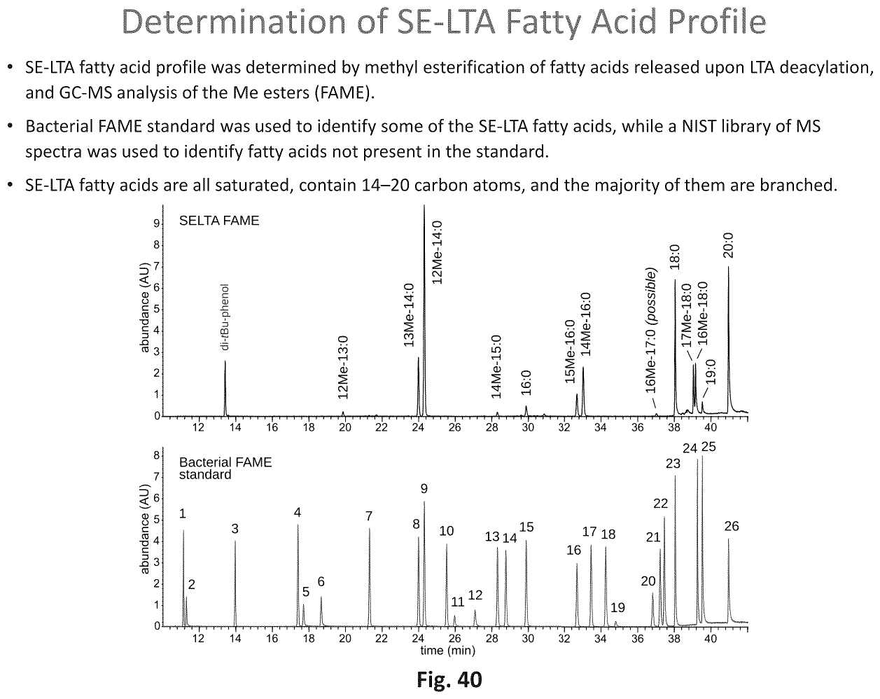

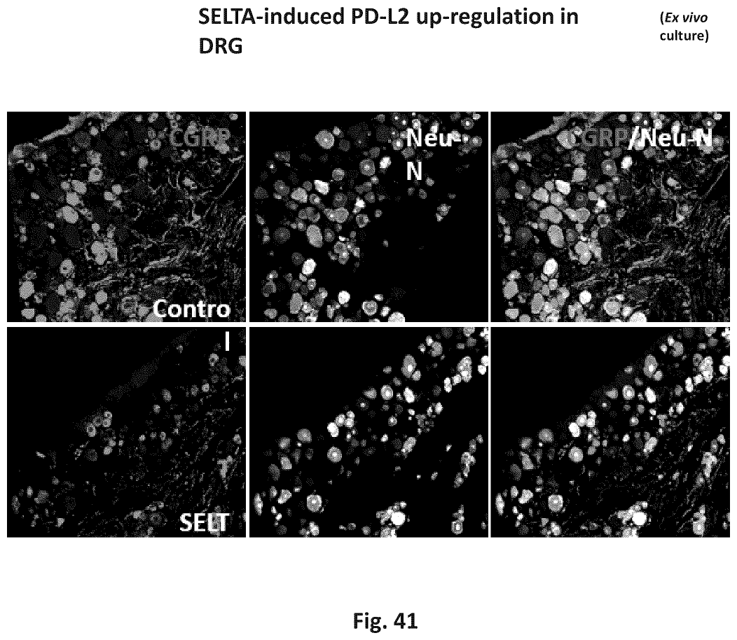

TABLE 3
FA residues, peaks in spectra, and abbreviations. See also FIG. 40.

| Peak | FA residue | Abbreviation |
|---|---|---|
| 1 | 12-methyl tridecanoate | 12Me-13:0 |
| 2 | 13-methyl tetradecanoate | 13Me-14:0 |
| 3 | 12-methyl tetradecanoate | 12Me-14:0 |
| 4 | 14-methyl pentadecanoate | 14Me-15:0 |
| 5 | hexadecanoate | 16:0 |
| 6 | 15-methyl hexadecanoate | 15Me-16:0 |
| 7 | 14-methyl hexadecanoate | 14Me-16:0 |
| 8 | 16-methyl heptadecanoate | 16Me-17:0 |
| 9 | octadecanoate | 18:0 |
| 10 | 17-methyl octadecanoate | 17Me-18:0 |
| 11 | 16-methyl octadecanoate | 16Me-18:0 |
| 12 | nonadecanoate | 19:0 |
| 13 | eicosanoate | 20:0 |

1. A method for treating pain in a subject in need thereof, the method comprising administering to the subject a composition comprising an effective amount of Staphylococcus epidermidis lipoteichoic acid (SELTA) for modulating the activity of the PD-1 pathway.

2. The method of claim 1, wherein the composition comprises live S. epidermidis bacteria.

3. The method of claim 1, wherein the composition comprises isolated SELTA.

4. The method of claim 3, wherein the isolated SELTA is derived from S. epidermidis bacteria.

5. The method of claim 3, wherein the isolated SELTA is synthesized.

6. The method of claim 1, wherein the SELTA comprises one or more chemical species of Formula I: 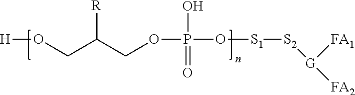
wherein n is 2-100; wherein each R is an amino acid, saccharide, or H; wherein S₁ and S₂ are monosaccharides connected by a linker; wherein G is a glyceride moiety; and wherein FA₁ and FA₂ are fatty acids.

7. The method of claim 6, wherein one or more R is D-alanine, a monosaccharide, or H.

8. The method of claim 6, wherein S₁ and S₂ are independently D-glucose, fructose, mannose, galactose, glucosamine, xylopyranose, or rhamnose, connected by linker selected from α(1→2), α(1→3), α(1→6), β(1→2), β(1→3) and β(→6).

9. The method of claim 6, wherein FA₁ and FA₂ are independently saturated or unsaturated fatty acids.

10. The method of claim 1, wherein the composition is administered orally.

11. The method of claim 1, wherein the subject is suffering from neurogenic pain.

12. The method of claim 1, wherein the subject has been diagnosed with multiple sclerosis, phantom limb, or is currently undergoing treatment with at least one chemotherapeutic agent or has been treated with at least one chemotherapeutic agent previously.

13. The method of claim 1, wherein the method modulates the expression of endogenous opioids in the subject.

14. A method of treating neurogenic pain in a subject in need thereof, the method comprising: administering a pharmaceutical composition comprising Staphylococcus epidermidis lipoteichoic acid (SELTA) to the subject to treat neurogenic pain in the subject.

15. The method of claim 14, wherein the subject has been diagnosed with multiple sclerosis, phantom limb, or is currently undergoing treatment with at least one chemotherapeutic agent or has been treated with at least one chemotherapeutic agent previously.

16. The method of claim 15, wherein the at least one chemotherapeutic agent comprises: a taxane, a platinum containing agent, a vinca alkaloid, an epothilone, eribulin, or bortezomib.

17. The method of claim 16, wherein the at least one chemotherapeutic agent comprises: paclitaxel or oxaliplatin.

18. A method of treating neurogenic inflammation in a subject in need thereof, the method comprising administering a pharmaceutical composition comprising Staphylococcus epidermidis lipoteichoic acid (SELTA) to the subject to treat neurogenic inflammation in the subject.

19. The method of claim 18, wherein the subject has been diagnosed with migraines, complex regional pain syndrome (CRPS), rheumatoid arthritis, or vulvodynia.

20. The method of claim 18, wherein the method reduces calcitonin related gene peptide (CRGP) in the subject.
